SEQUENCE LISTING (95 sequences)

SEQ ID NO: 1 - protein, 26 aa, Apis florea
GIGAILKVLATGLPTLISWIKNKRKQ

SEQ ID NO: 2 - protein, 26 aa, Artificial Sequence
  Melittin-like peptide
AIGAILKVLATGLPTLISWIKNKRKQ

SEQ ID NO: 3 - protein, 26 aa, Artificial Sequence
  Melittin-like peptide
CIGAILKVLATGLPTLISWIKNKRKQ

SEQ ID NO: 4 - protein, 26 aa, Artificial Sequence
  Melittin-like peptide
FIGAILKVLATGLPTLISWIKNKRKQ

SEQ ID NO: 5 - protein, 26 aa, Artificial Sequence
  Melittin-like peptide
HIGAILKVLATGLPTLISWIKNKRKQ

SEQ ID NO: 6 - protein, 26 aa, Artificial Sequence
  Melittin-like peptide
IIGAILKVLATGLPTLISWIKNKRKQ

SEQ ID NO: 7 - protein, 26 aa, Artificial Sequence
  Melittin-like peptide
LIGAILKVLATGLPTLISWIKNKRKQ

SEQ ID NO: 8 - protein, 26 aa, Artificial Sequence
  Melittin-like peptide
  misc_feature (1)..(1): Xaa is Nle
XIGAILKVLATGLPTLISWIKNKRKQ

SEQ ID NO: 9 - protein, 26 aa, Artificial Sequence
  Melittin-like peptide
VIGAILKVLATGLPTLISWIKNKRKQ

SEQ ID NO: 10 - protein, 26 aa, Artificial Sequence
  Melittin-like peptide
WIGAILKVLATGLPTLISWIKNKRKQ

SEQ ID NO: 11 - protein, 26 aa, Artificial Sequence
  Melittin-like peptide
YIGAILKVLATGLPTLISWIKNKRKQ

SEQ ID NO: 12 - protein, 26 aa, Artificial Sequence
  D-form Apis florea melittin sequence
GIGAILKVLACGLPTLISWIKNKRKQ

SEQ ID NO: 13 - protein, 26 aa, Artificial Sequence
  Melittin-like peptide
GIGAILKVLATLLPTLISWIKNKRKQ

SEQ ID NO: 14 - protein, 26 aa, Artificial Sequence
  Melittin-like peptide
GIGAILKVLATWLPTLISWIKNKRKQ

SEQ ID NO: 15 - protein, 26 aa, Artificial Sequence
  Melittin-like peptide
GIGAILKVLATGLPTLISWIKTKRKQ

SEQ ID NO: 16 - protein, 26 aa, Artificial Sequence
  Melittin-like peptide
YIGAILNVLATGLPTLISWIKNKRKQ

SEQ ID NO: 17 - protein, 26 aa, Artificial Sequence
  Melittin-like peptide
YIGAILAVLATGLPTLISWIKNKRKQ

SEQ ID NO: 18 - protein, 26 aa, Artificial Sequence
  Melittin-like peptide
LIGAILSVLATGLPTLISWIKNKRKQ

SEQ ID NO: 19 - protein, 26 aa, Artificial Sequence
  Melittin-like peptide
LIGAILRVLATGLPTLISWIKNKRKQ

SEQ ID NO: 20 - protein, 26 aa, Artificial Sequence
  Melittin-like peptide
LIGAILHVLATGLPTLISWIKNKRKQ

SEQ ID NO: 21 - protein, 26 aa, Artificial Sequence
  Melittin-like peptide
LIGAILKVLACGLPTLISWIKNKRKQ

SEQ ID NO: 22 - protein, 26 aa, Artificial Sequence
  Melittin-like peptide
LIGAILKVLATLLPTLISWIKNKRKQ

SEQ ID NO: 23 - protein, 26 aa, Artificial Sequence
  Melittin-like peptide
YIGAILKVLATGLLTLISWIKNKRKQ

SEQ ID NO: 24 - protein, 26 aa, Artificial Sequence
  Melittin-like peptide
LIGAILKVLATGLPCLISWIKNKRKQ

SEQ ID NO: 25 - protein, 26 aa, Artificial Sequence
  Melittin-like peptide
LIGAILKVLATGLPTLICWIKNKRKQ

SEQ ID NO: 26 - protein, 26 aa, Artificial Sequence
  Melittin-like peptide
YIGAILKVLATGLPTLISAIKNKRKQ

SEQ ID NO: 27 - protein, 26 aa, Artificial Sequence
  Melittin-like peptide
GIGAILKVLACGLPTLISWLKNKRKQ

SEQ ID NO: 28 - protein, 26 aa, Artificial Sequence
  Melittin-like peptide
YIGAILKVLATGLPTLISWIANKRKQ

SEQ ID NO: 29 - protein, 26 aa, Artificial Sequence
  Melittin-like peptide
YIGAILKVLATGLPTLISWIKNARKQ

SEQ ID NO: 30 - protein, 26 aa, Artificial Sequence
  Melittin-like peptide
LIGAILKVLATGLPTLISWIKNKAKQ

SEQ ID NO: 31 - protein, 26 aa, Artificial Sequence
  Melittin-like peptide
YIGAILKVLATGLPTLISWIKNKRAQ

SEQ ID NO: 32 - protein, 26 aa, Artificial Sequence
  Melittin-like peptide
YIGAILKVLATGLPTLISWIKNKRKC

SEQ ID NO: 33 - protein, 26 aa, Artificial Sequence
  Melittin-like peptide
LLGAILKVLACGLPTLISWIKNKRKQ

SEQ ID NO: 34 - protein, 26 aa, Artificial Sequence
  Melittin-like peptide
LIGALLKVLACGLPTLISWIKNKRKQ

SEQ ID NO: 35 - protein, 26 aa, Artificial Sequence
  Melittin-like peptide
YIGAILAVLATGLPTLISWIANKRKQ

SEQ ID NO: 36 - protein, 26 aa, Artificial Sequence
  Melittin-like peptide
YIGAILAVLATGLPTLISWIKNARKQ

SEQ ID NO: 37 - protein, 26 aa, Artificial Sequence
  Melittin-like peptide
LIGAILKVLACGLPTLLSWIKNKRKQ

SEQ ID NO: 38 - protein, 26 aa, Artificial Sequence
  Melittin-like peptide
LIGAILKVLACGLPTLICWIKNKRKQ

SEQ ID NO: 39 - protein, 26 aa, Artificial Sequence
  Melittin-like peptide
GIGAILKVLACGLPGLIGWIKNKRKQ

SEQ ID NO: 40 - protein, 26 aa, Artificial Sequence
  Melittin-like peptide
GIGAILKVLACGLPALIAWIKNKRKQ

SEQ ID NO: 41 - protein, 26 aa, Artificial Sequence
  Melittin-like peptide
YIGAILAVLATGLPTLISWIANARKQ

SEQ ID NO: 42 - protein, 26 aa, Artificial Sequence
  Melittin-like peptide
YIAAILKVLAAALATLISWIKNKRKQ

SEQ ID NO: 43 - protein, 26 aa, Artificial Sequence
  Melittin-like peptide
LLGALLKVLATGLPTLLSWLKNKRKQ

SEQ ID NO: 44 - protein, 26 aa, Artificial Sequence
  Melittin-like peptide
  misc_feature 2, 5, 17, 20: Xaa is Nle
LXGAXLKVLATGLPTLXSWXKNKRKQ

SEQ ID NO: 45 - protein, 26 aa, Artificial Sequence
  Melittin-like peptide
LVGAVLKVLATGLPTLVSWVKNKRKQ

SEQ ID NO: 46 - protein, 26 aa, Artificial Sequence
  Melittin-like peptide
GLGALLKVLACGLPTLLSWLKNKRKQ

SEQ ID NO: 47 - protein, 26 aa, Artificial Sequence
  Melittin-like peptide
  misc_feature 2, 5, 17, 20: Xaa is Nle
GXGAXLKVLACGLPTLXSWXKNKRKQ

SEQ ID NO: 48 - protein, 31 aa, Artificial Sequence
  Melittin-like peptide
CEDDLLLGAILKVLATGLPTLISWIKNKRKQ

SEQ ID NO: 49 - protein, 31 aa, Artificial Sequence
  Melittin-like peptide
CLVVLIVVAILKVLATGLPTLISWIKNKRKQ

SEQ ID NO: 50 - protein, 26 aa, Apis mellifera
GIGAVLKVLTTGLPALISWIKRKRQQ

SEQ ID NO: 51 - protein, 27 aa, Artificial Sequence
  Melittin-like peptide
CLIGAILKVLATGLPTLISWIKNKRKQ

SEQ ID NO: 52 - protein, 27 aa, Artificial Sequence
  Melittin-like peptide
  misc_feature (2)..(2): Xaa is Nle
CXIGAILKVLATGLPTLISWIKNKRKQ

SEQ ID NO: 53 - protein, 27 aa, Artificial Sequence
  Melittin-like peptide
GLIGAILKVLATGLPTLISWIKNKRKQ

SEQ ID NO: 54 - protein, 27 aa, Artificial Sequence
  Melittin-like peptide
LLIGAILKVLATGLPTLISWIKNKRKQ

SEQ ID NO: 55 - protein, 29 aa, Artificial Sequence
  Melittin-like peptide
KLKLIGAILKVLATGLPTLISWIKNKRKQ

SEQ ID NO: 56 - protein, 29 aa, Artificial Sequence
  Melittin-like peptide
KLKYIGAILKVLATGLPTLISWIKNKRKQ

SEQ ID NO: 57 - protein, 30 aa, Artificial Sequence
  Melittin-like peptide
CKLKLIGAILKVLATGLPTLISWIKNKRKQ

SEQ ID NO: 58 - protein, 30 aa, Artificial Sequence
  Melittin-like peptide
  misc_feature (5)..(5): Xaa is Nle
CKLKXIGAILKVLATGLPTLISWIKNKRKQ

SEQ ID NO: 59 - protein, 30 aa, Artificial Sequence
  Melittin-like peptide
GKLKLIGAILKVLATGLPTLISWIKNKRKQ

SEQ ID NO: 60 - protein, 30 aa, Artificial Sequence
  D-form Melittin-like peptide
CPANLIGAILKVLATGLPTLISWIKNKRKQ

SEQ ID NO: 61 - protein, 31 aa, Artificial Sequence
  Melittin-like peptide
DEPLRAIGAILKVLATGLPTLISWIKNKRKQ

SEQ ID NO: 62 - protein, 27 aa, Artificial Sequence
  Melittin-like peptide
GIGAILKVLATGLPTLISWIKNKRKQC

SEQ ID NO: 63 - protein, 27 aa, Artificial Sequence
  Melittin-like peptide
LIGAILKVLATGLPTLISWIKNKRKQC

SEQ ID NO: 64 - protein, 27 aa, Artificial Sequence
  Melittin-like peptide
  misc_feature (1)..(1): Xaa is Nle
XIGAILKVLATGLPTLISWIKNKRKQC

SEQ ID NO: 65 - protein, 30 aa, Artificial Sequence
  Melittin-like peptide
LIGAILKVLATGLPTLISWIKNKRKQKLKC

SEQ ID NO: 66 - protein, 34 aa, Artificial Sequence
  Melittin-like peptide
YIGAILKVLATGLPTLISWIKNKRKQPLGIAGQC

SEQ ID NO: 67 - protein, 31 aa, Artificial Sequence
  Melittin-like peptide
LIGAILKVLATGLPTLISWIKNKRKQKKKKK

SEQ ID NO: 68 - protein, 31 aa, Artificial Sequence
  Melittin-like peptide
YIGAILKVLATGLPTLISWIKNKRKQGFKGC

SEQ ID NO: 69 - protein, 30 aa, Artificial Sequence
  Melittin-like peptide
CFKLIGAILKVLATGLPTLISWIKNKRKQC

SEQ ID NO: 70 - protein, 25 aa, Artificial Sequence
  Melittin-like peptide
FGAILKVLATGLPTLISWIKNKRKQ

SEQ ID NO: 71 - protein, 23 aa, Artificial Sequence
  Melittin-like peptide
LIGAILKVLATGLPTLISWIKNK

SEQ ID NO: 72 - protein, 21 aa, Artificial Sequence
  Melittin-like peptide
LIGAVLKVLTTGLPALISWIK

SEQ ID NO: 73 - protein, 23 aa, Artificial Sequence
  Melittin-like peptide
LIGAVLKVLTTGLPALISWIKGE

SEQ ID NO: 74 - protein, 26 aa, Artificial Sequence
  Melittin-like peptide
QKRKNKIWSILTPLGTALVKLIAGIL

SEQ ID NO: 75 - protein, 29 aa, Artificial Sequence
  Melittin-like peptide
KLKQKRKNKIWSILTPLGTALVKLIAGIL

SEQ ID NO: 76 - protein, 27 aa, Artificial Sequence
  Melittin-like peptide
GIGAVLKVLTTGLPALISWISRKKRQQ

SEQ ID NO: 77 - protein, 25 aa, Artificial Sequence
  Melittin-like peptide
GIGARLKVLTTGLPRISWIKRKRQQ

SEQ ID NO: 78 - protein, 26 aa, Artificial Sequence
  Melittin-like peptide
GIGAILKVLSTGLPALISWIKRKRQE

SEQ ID NO: 79 - protein, 26 aa, Artificial Sequence
  Melittin-like peptide
GIGAVLKVLTTGLPALIGWIKRKRQQ

SEQ ID NO: 80 - protein, 26 aa, Artificial Sequence
  Melittin-like peptide
GIGAVLKVLATGLPALISWIKRKRQQ

SEQ ID NO: 81 - protein, 26 aa, Artificial Sequence
  Melittin-like peptide
GIGAVLKVLSTGLPALISWIKRKRQQ

SEQ ID NO: 82 - protein, 26 aa, Artificial Sequence
  Melittin-like peptide
GIGAILRVLATGLPTLISWIKNKRKQ

SEQ ID NO: 83 - protein, 26 aa, Artificial Sequence
  Melittin-like peptide
GIGAILKVLATGLPTLISWIKRKRKQ

SEQ ID NO: 84 - protein, 26 aa, Artificial Sequence
  Melittin-like peptide
GIGAILKVLATGLPTLISWIKKKKQQ

SEQ ID NO: 85 - protein, 32 aa, Artificial Sequence
  Melittin-like peptide
GIGAILKVLATGLPTLISWIKNKRKQGSKKKK

SEQ ID NO: 86 - protein, 28 aa, Artificial Sequence
  Melittin-like peptide
KKGIGAILKVLATGLPTLISWIKNKRKQ

SEQ ID NO: 87 - protein, 26 aa, Artificial Sequence
  Melittin-like peptide
GIGAILEVLATGLPTLISWIKNKRKQ

SEQ ID NO: 88 - protein, 24 aa, Artificial Sequence
  Melittin-like peptide
GIGAVLKVLTTGLPALISWIKRKR

SEQ ID NO: 89 - protein, 22 aa, Artificial Sequence
  Melittin-like peptide
GIGAVLKVLTTGLPALISWIKR

SEQ ID NO: 90 - protein, 26 aa, Artificial Sequence
  Melittin-like peptide
CIGAVLKVLTTGLPALISWIKRKRQQ

SEQ ID NO: 91 - protein, 26 aa, Artificial Sequence
  Melittin-like peptide
QQRKRKIWSILAPLGTTLVKLVAGIG

SEQ ID NO: 92 - protein, 26 aa, Artificial Sequence
  Melittin-like peptide
QQRKRKIWSILAPLGTTLVKLVAGIC

SEQ ID NO: 93 - protein, 26 aa, Artificial Sequence
  Melittin-like peptide
QQKKKKIWSILAPLGTTLVKLVAGIC

SEQ ID NO: 94 - protein, 26 aa, Artificial Sequence
  Melittin-like peptide
QKRKNKIWSILTPLGTALVKLIAGIG

SEQ ID NO: 95 - protein, 26 aa, Artificial Sequence
  Melittin-like peptide
QQRKRKIWSILAALGTTLVKLVAGIC

(19) United States
(12) Patent Application Publication
(10) Pub. No.: US 2018/0312537 A1
(43) Pub. Date: Nov. 1, 2018

(54) METHOD OF MODIFYING A PEPTIDE

(71) Applicant: Arrowhead Pharmaceuticals, Inc., Pasadena, CA (US)

(72) Inventors: Tao Pei, Monona, WI (US); Zhen Li, Monona, WI (US); Agnieszka Glebocka, Madison, WI (US); Andrew Zhou, Monona, WI (US)

(21) Appl. No.: 16/032,172

(22) Filed: Jul. 11, 2018

Related U.S. Application Data
(63) Continuation of application No. PCT/US17/13643, filed on Jan. 16, 2017.
(60) Provisional application No. 62/278,620, filed on Jan. 14, 2016.

(51) Int. Cl. C07K 1/107; C07D 207/20; C07K 14/435

(52) U.S. Cl. CPC C07K 1/1077; C07D 213/06; C07K 14/435; C07D 207/20

(57) ABSTRACT

Described herein are methods of modifying a peptide with a chemical agent. The peptide may be an amphipathic peptide, such as a melittin-like peptide. The reactants are dissolved in an organic solvent and the reaction is allowed to proceed during sequential additions of a first base and a second base. Reaction in organic solvent with sequential base additions yields greater than 90% modification of both N-terminal and internal primary amine modification.

### CROSS-REFERENCE TO RELATED APPLICATIONS

This application is a continuation of International Patent Application No. PCT/US17/13643, with an international filing date of 16 Jan. 2017, designating the United States, which claims priority to and is based on U.S. Provisional Patent Application No. 62/278,620, filed 14 Jan. 2016, the contents of each of which are incorporated herein by reference.

### BACKGROUND

Peptides and other biologically-derived molecules are often incorporated into medical and biological products. In many cases, in order to provide the desired physical and biological properties for their intended use, peptides require modification with one or more chemical agents. It is frequently the case that the amines (the N-terminal amine and/or internal amines such as lysine ε-amines) of a peptide are sites where modification with a chemical agent is desirable. Amine modification of peptides must be efficient and reliable, especially for peptides used in biopharmaceutical products. However, efficient amine modification of a peptide is often interfered by the peptide folding of itself or its intermediate.

One example of a particular use for modified peptides is as a vehicle that aids in the delivery of oligonucleotides to cells in vivo. It has been shown that certain peptides, such as melittin and melittin-like-peptides (together MLPs), can facilitate the entry of oligonucleotides into cells (see, e.g., U.S. Pat. No. 8,501,930). MLPs are small amphipathic membrane active peptides comprising about 23 to about 32 amino acids that are derived from the naturally occurring bee venom peptide melittin. MLPs are predominantly hydrophobic on the amino terminal ends and predominantly hydrophilic (cationic) on the carboxyl terminal ends. They are potent membrane active peptides capable of disrupting plasma membranes or lysosomal/endocytic membranes. When used as a delivery peptide, the MLP amine groups, an N-terminal amine and multiple lysine ε-amines, must be reversibly modified with chemical agents to limit potential toxicity in vivo. Dimethylmaleic anhdydrides and dipeptide-amindobenzylcarbonates are examples of chemical agents that may be used to modify MLPs. The modification of MLPs using these chemical agents reversibly inhibits membrane activity of the MLP until the MLP reaches an endocytic cellular vesicle.

While modification of peptide amines is desirable, traditional methods used for modification of peptides with chemical agents is highly inefficient. In the case of peptides such as MLPs, for example, modification in a typical aqueous solution requires 5:1 (wt/wt) equivalents of CDM-NAG to MLP to achieve 90% modification (U.S. Pat. No. 8,501,930). In the case of MLPs, it was found that lysine ε-amine modification often leads to poor N-terminal amine modification (U.S. Pat. No. 8,501,930). Further, the relatively dilute peptide concentration in aqueous solution necessary for ≥90% modification limits batch size at manufacturing site. Higher concentration in aqueous buffer causes a decrease in conjugation efficiency. Modification in aqueous solution was also found to lead to inconsistent and sometimes low conjugation efficiency on N-terminal and/or internal amines.

Modification of peptides in organic solvent leads to significant advantages over generally applied aqueous conditions. However, due the likelihood of aggregation or “gelling” of peptides in organic solvent, it is not trivial to develop a chemistry modification under non-aqueous conditions. We now provide new methods of peptide modification that achieve high efficiency of both N-terminal and internal amine modification. The high concentration of peptide under the described conditions significantly improves productivity at manufacture scale.

### SUMMARY

Disclosed herein are methods of modifying a peptide with a chemical agent comprising a reactive moiety. The methods disclosed herein comprise dissolving the peptide and the chemical agent in an organic solvent and incubating until at least 90% of the N-terminal amines of the peptide are modified with the chemical agent.

The organic solvent used in the methods disclosed herein can be trifluoroethanol (TFE), hexafluoro-iso-propanol (HFIP), dimethyl sulfoxide (DMSO), mixtures of TFE, HFIP, and DMSO, mixtures of TFE, HFIP, or DMSO with one or more miscible solvents. In some embodiments, the organic solvent is TFE.

Peptides suitable for the disclosed methods include peptides that are sufficiently soluble in the selected organic solvent. In some embodiments, the peptide used in the methods disclosed herein is an amphipathic peptide. In some embodiments, the peptide used in the methods disclosed herein is a melittin-like peptide (MLP). In some embodiments, the MLP is selected from any of SEQ ID NOs: 1-95 disclosed herein. The peptides suitable for the disclosed methods may be between about 5 and about 40 amino acids if single stranded, or between about 5 and about 80 amino acids if the peptide is a dimer or is double stranded. The methods described herein may also be used to modify biologically-derived molecules or polymers having properties similar to the peptides disclosed as suitable for the disclosed methods.

In some embodiments, the methods include dissolving the peptide, the chemical agent, and a first base in an organic solvent, and incubating until at least 90% of the N-terminal amines of the peptide are modified. The optional first base, when added, can be pyridine (Py), pyridine with substitution on the aromatic ring, heterocycle, N-methyl morpholine, N-methyl imidazole, alkylamine, Tris, HEPES, MOPS, triethylamine, or dimethylaminopyridine (DMAP). The addition of the first base can improve the efficiency of the reaction and provide for 90% of the N-terminal amines of the peptide to be modified. In some embodiments, the first base is pyridine.

In some embodiments, the methods disclosed herein include dissolving the peptide, the chemical agent, and a first base, and a miscible solvent in an organic solvent. The optional added miscible solvent, when added, can be alcohol, methanol (MeOH), ethanol, (EtOH), n-propanol, n-butanol, ethylene glycol, dimethylformamide (DMF), dimethylacetamide (DMAc), N-methyl-2-pyrrolidone (NMP), halogenated solvent, chloroform, dichloromethane, dichloroethane, ether, diethyl ether, tetrahydrofuran (THF), methyl tert-butyl ether (MTBE), glycol ethers (glyme), dimethoxyethane, bis-2-methoxyethyl ether (diglyme), ester, ethyl acetate, isopropyl acetate, or nitrile (MeCN). The addition of the first base and a miscible solvent can improve the efficiency of the reaction and provide for 90% of the N-terminal amines of the peptide to be modified.

In some embodiments, the methods disclosed herein further comprise adding a second base after after 90% of the N-terminal amines have been modified. The addition of the second base followed by incubation provides for at least 90% of the total primary amines of the peptide to be modified with the chemical agent. In some embodiments, the addition of the second base followed by incubation provides for at least 95%, at least 98%, or at least 99% modification of the total primary amines of the peptide. In some embodiments, the second base is an organic base. In some embodiments, the second base is a trialkylamine, such as trimethylamine (TEA).

The organic solvent is selected to have high solubility of the peptide, the chemical agent comprising a reactive moiety or “modifier”, and the resulting modified peptide. When added, the first base is an organic base that is added such that the solution achieves a pH in water greater than 5 and less than 9. The second base is an organic base that is added such that the solution achieves a pH in water of greater than or equal to 9.

The reactive moiety of the chemical agent can be any moiety that is known to react with an amine. For example, the reactive moiety of the chemical agent may be comprised of (i) an acid (or its activated form, such as an acid chloride, acid anhydride, or ester); (ii) a ketone or aldehyde; (iii) an epoxide; (iv) an alkyl halide; (v) an activated alcohol; or (iv) a combination of any of (i) through (v). In some embodiments, the reactive moiety is an acid anhydride. In some embodiments, the chemical agent comprises a substituted maleic anhydride. In some embodiments, the chemical agent comprises an asialoglycoprotein receptor ligand. In some embodiments, the chemical agent comprises a polyethylene glycol.

The methods disclosed herein provide for the modified peptide to be precipitated. The modified peptide may be precipitated by reverse addition of an antisolvent. The antisolvent may be comprised of MeCN, ethyl acetate, acetonitrile, isopropyl acetate, esters, or ethers. The methods disclosed herein further provide for the precipitated peptide to be purified by tangential flow filtration. The methods disclosed herein further provide for the purified modified peptide to be lyophilized.

In some embodiments, the methods disclosed herein provide for the ratio of chemical agent to peptide to be ≥1.4:1, ≥1.5:1, ≥1.6:1, ≥1.7:1, 1.4-5:1, 1.4-2.0:1, 1.4-1.9:1, 1.4-1.8:1, 1.4-1.7:1, 1.4-1.6:1, 1.4-1.5:1, 1.5-2:1, 1.5-1.9:1, 1.5-1.8:1, 1.5-1.7:1, 1.5-1.6:1, 1.6-2:1, 1.6-1.9:1, 1.6-1.8:1, or 1.6-1.7:1.

In some embodiments, the methods described herein are directed to syntheses of melittin-like peptide-N-acetylgalactoseaminen (MLP-NAGₙ or MLP-NAG or modified MLP) comprising: reacting melittin-like peptide (MLP) with CDM-NAG in organic solvent in the sequential presence of a first base and a second base. In some embodiments, the syntheses results in the conjugate CDM-NAGs to MLP primary amines to yield MLP-NAGₙ wherein n is an integer from 1 to the number of primary amines on the MLP. For GILL MLP, which contains five primary amines (four lysine amines and one N-terminal amine), n=an integer between 1 and 5. The formed MLP-NAGₙ conjugate can be a mixture of MLP-NAG₁, MLP-NAG₂, MLP-NAG₃, MLP-NAG₄, . . . MLP-NAGₙ (i.e. n=1, 2, 3, 4, . . . n), a mixture of MLP-NAG₂, MLP-NAG₃, MLP-NAG₄, MLP-NAGₙ, a mixture of MLP-NAG₃, MLP-NAG₄, MLP-NAGₙ, etc. In some embodiments, for an MLP having five primary amines, the formed MLP-NAGₙ conjugate can be a mixture of MLP-NAG₁, MLP-NAG₂, MLP-NAG₃, MLP-NAG₄, and MLP-NAG₅. In some embodiments, for an MLP having five primary amines, the formed MLP-NAGₙ conjugate can be a mixture of MLP-NAG₂, MLP-NAG₃, MLP-NAG₄, and MLP-NAG₅. In some embodiments, for an MLP having five primary amines, the formed MLP-NAGₙ conjugate can be a mixture of MLP-NAG₃, MLP-NAG₄, and MLP-NAG₅. In some embodiments, for an MLP having five primary amines, the formed MLP-NAGₙ conjugate can be a mixture of MLP-NAG₄ and MLP-NAG₅. The described method facilitates at least 90% modification of MLP primary amines.

In some embodiments, the methods described herein are directed to syntheses of MLP-NAG comprising: reacting a plurality of melittin-like peptide (MLP) with a plurality of CDM-NAG in trifluoroethanol in the sequential presence of pyridine and triethylamine to yield MLP-NAGₙ wherein n=an integer between 1 and 5. The formed MLP-NAGₙ conjugate can be a mixture of MLP-NAG₁, MLP-NAG₂, MLP-NAG₃, MLP-NAG₄, and MLP-NAG₅ (i.e. n=1, 2, 3, 4, and 5). In some embodiments, the formed MLP-NAGₙ conjugate can be a mixture of MLP-NAG₂, MLP-NAG₃, MLP-NAG₄, and MLP-NAG₅. In some embodiments, the formed MLP-NAGₙ conjugate can be a mixture of MLP-NAG₃, MLP-NAG₄, and MLP-NAG₅. In some embodiments, the formed MLP-NAGₙ conjugate can be a mixture of MLP-NAG₄, and MLP-NAG₅. The described method facilitates at least 90% modification of MLP primary amines. For MLP peptides having more than 5 primary amines, n is an integer from 1 to the number of primary amines on the MLP.

In some embodiments, the methods described herein are directed to syntheses of MLP-NAG comprising: dissolving MLP salt (such as MLP acetate) and CDM-NAG in trifluoroethanol, incubating the reactants until at least 90% of N-terminal amines are modified, adding triethylamine, and incubating the reactants until at least 90% of all primary amines are modified to yield MLP-NAGₙ wherein n=an integer between 1 and 5. The formed MLP-NAGₙ conjugate can be a mixture of MLP-NAG₁, MLP-NAG₂, MLP-NAG₃, MLP-NAG₄, and MLP-NAG₅ (i.e. n=1, 2, 3, 4, and 5). In some embodiments, the formed MLP-NAGₙ conjugate can be a mixture of MLP-NAG₂, MLP-NAG₃, MLP-NAG₄, and MLP-NAG₅. In some embodiments, the formed MLP-NAGₙ conjugate can be a mixture of MLP-NAG₃, MLP-NAG₄, and MLP-NAG₅. In some embodiments, the formed MLP-NAGₙ conjugate can be a mixture of MLP-NAG₄ and MLP-NAG₅. The described method facilitates at least 90% modification of MLP primary amines. For MLP peptides having more than 5 primary amines, n is an integer from 1 to the number of primary amines on the MLP.

In some embodiments, the methods described herein are directed to syntheses of MLP-NAG comprising: reacting MLP Peptide with CDM-NAG in trifluoroethanol in the sequential presence of pyridine and triethylamine to yield MLP-NAGₙ wherein n=an integer between 1 and 5. The formed MLP-NAGₙ conjugate can be a mixture of MLP-NAG₁, MLP-NAG₂, MLP-NAG₃, MLP-NAG₄, and MLP-NAG₅ (i.e. n=1, 2, 3, 4, and 5). In some embodiments, the formed MLP-NAGₙ conjugate can be a mixture of MLP-NAG₂, MLP-NAG₃, MLP-NAG₄, and MLP-NAG₅. In some embodiments, the formed MLP-NAGₙ conjugate can be a mixture of MLP-NAG₃, MLP-NAG₄, and MLP-NAG₅. In some embodiments, the formed MLP-NAGₙ conjugate can be a mixture of MLP-NAG₄ and MLP-NAG₅. The mixture MLP-NAGₙ is then precipitated by reverse addition into antisolvent to yield the conjugate solid. The antisolvent is selected to have low solubility of the MLP-NAG. For MLP peptides having more than 5 primary amines, n is an integer from 1 to the number of primary amines on the MLP.

In some embodiments, the methods described herein are directed to syntheses of MLP-NAG comprising: reacting MLP with CDM-NAG in the presence of pyridine and triethylamine in trifluoroethanol to yield MLP-NAGₙ wherein n=an integer between 1 and 5. The formed MLP-NAGₙ conjugate can be a mixture of MLP-NAG₁, MLP-NAG₂, MLP-NAG₃, MLP-NAG₄, and MLP-NAG₅ (i.e. n=1, 2, 3, 4, and 5). In some embodiments, the formed MLP-NAGₙ conjugate can be a mixture of MLP-NAG₂, MLP-NAG₃, MLP-NAG₄, and MLP-NAG₅. In some embodiments, the formed MLP-NAGₙ conjugate can be a mixture of MLP-NAG₃, MLP-NAG₄, and MLP-NAG₅. In some embodiments, the formed MLP-NAGₙ conjugate can be a mixture of MLP-NAG₄ and MLP-NAG₅. The mixture MLP-NAGₙ is then precipitated by reverse addition into antisolvent to yield the conjugate solid. The MLP-NAG is then purified by Tangential Flow Filtration (TFF) and formulated for in vivo administration. For MLP peptides having more than 5 primary amines, n is an integer from 1 to the number of primary amines on the MLP.

The methods described herein can be readily used to modify peptides for which the peptide, the chemical agent comprising a reactive group, and the modified peptide are all sufficiently soluble in the organic solvent. The method for synthesis is particularly useful for peptides with one or more primary amines. The methods for synthesis disclosed herein provide for the modification of at least 90%, at least 95%, at least 98%, or at least 99% of the primary amines of the peptide.

In some embodiments, the MLP-NAG synthesized by the described methods can be used to deliver RNA interference (RNAi) polynucleotides to liver cells in vivo. The delivered RNAi polynucleotides can be delivered for the purpose of inhibiting gene expression. The inhibition of gene expression can have a therapeutic effect.

### BRIEF DESCRIPTION OF THE DRAWINGS

FIG. 1. Diagram illustrating the reaction mechanism for the preparation of MLP-NAG₅, using the methods disclosed herein.

FIG. 2. HPLC chromatogram depicting N-terminal amine modification (see, e.g., Example 1).

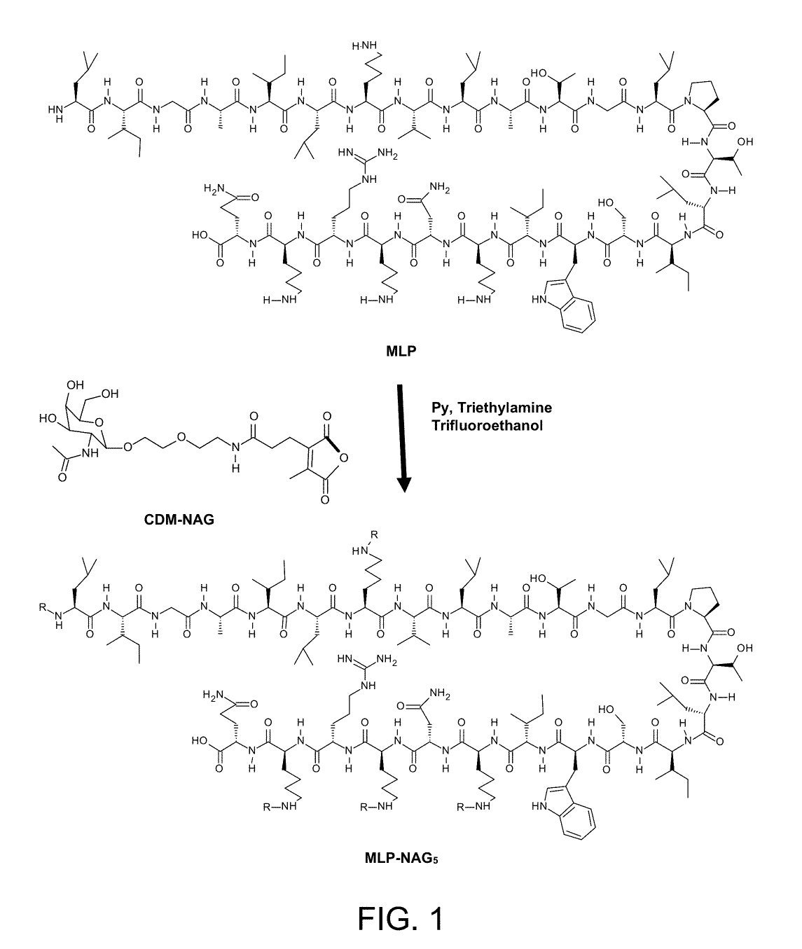

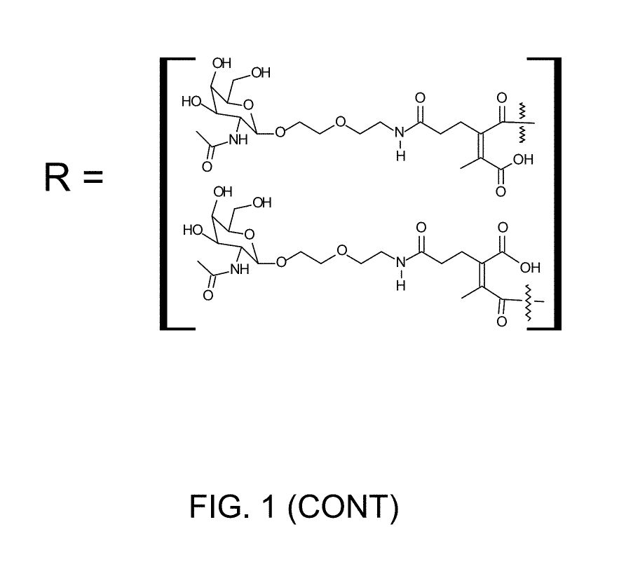

### DETAILED DESCRIPTION

Described herein are improved methods of peptide modification that provide higher modification yield and consistency than previously described aqueous methods. We show that modification of peptides in organic solvent provides significant advantages over previously described modification in aqueous conditions. The described methods provide improved consistency and conjugation efficiency on N-terminal amines, internal amines (e.g., lysine c amines), or both. The methods described herein are readily used to modify peptides, including amphipathic peptides, with chemical agents comprising a reactive moiety. The methods described herein may be readily used with any peptides and chemical agents comprising a reactive moiety that are sufficiently soluble in organic solvent to facilitate the modification of the peptide.

The described methods also provide for increased conjugation at increased concentration which is more suitable for larger scale production. High concentration of peptide under conjugation conditions allows high productivity during scale-up and significantly improves productivity at manufacture scale.

In some embodiments, the peptide selected for use in the methods disclosed herein is an amphipathic peptide. In some embodiments, the peptide is a melittin-like peptide (MLP). In some embodiments, the disclosed methods provide for the reaction of melittin-like peptide (MLP) with CDM-NAG in the presence of pyridine and triethylamine in trifluoroethanol, which provides a more complete reaction and higher yield of MLP-NAG than was previously available using the prior described conjugate methods. The process disclosed herein yields consistently higher primary amine modification rate on both N-terminal amine and internal amines (e.g., lysine G-amines). Furthermore, high solubility of MLP in trifluoroethanol allows high manufacture efficiency including upon scale-up (kg quantities). The described conjugation methods also enable isolation of conjugate as a solid, which improves stability during storage and subsequent handling. The disclosed methods are generally applicable to a wide variety of peptides or other biologically-derived molecules or polymers, including amphipathic peptides such as MLPs.

For example, in some embodiments, the peptide may be a single stranded peptide comprising between about 5 and about 40 amino acids. In some embodiments, the peptide is a single stranded peptide comprising between about 10 and about 35 amino acids. In some embodiments, the peptide is a single stranded peptide comprising between about 15 and about 35 amino acids. In some embodiments, the peptide is a single stranded peptide comprising between about 20 and about 35 amino acids. In some embodiments, the peptide is a single stranded peptide comprising between about 23 and about 32 amino acids. In some embodiments, the peptide may be a dimer or double stranded peptide (such as, for example, insulin), and be comprised of between about 5 and about 80 amino acids.

### Organic Solvent

The use of organic solvent enables conjugation of peptide and chemical agent at high concentration, ≥50, ≥70, ≥100 g/L peptide. Further, we have found that both peptides, and in particular amphipathic peptides, as well as certain chemical agents comprising a reactive moiety such as CDM-NAG, are stable in certain organic solvents. For example, reacting MLP with CDM-NAG in organic solvent allows efficient conjugation, with greater that ≥90, ≥95, ≥98, or ≥99% primary amine modification, using are little as 1.4-5, 1.4-2.0, 1.4-1.9, 1.4-1.8, 1.4-1.7, 1.4-1.6, 1.4-1.5 molar equivalents of CDM-NAG. Suitable organic solvents include, but are not limited to: trifluoroethanol (TFE), Hexafluoro-iso-propanol (HFIP), dimethyl sulfoxide (DMSO), and mixtures of TFE, HFIP, and DMSO. Suitable organic solvents also include mixtures of TFE, HFIP, or DMSO with other miscible solvents, including, but not limited to: alcohol, methanol (MeOH), ethanol, (EtOH), n-propanol, n-butanol, ethylene glycol, dimethylformamide (DMF), dimethylacetamide (DMAc), N-methyl-2-pyrrolidone (NMP), halogenated solvent, chloroform, dichloromethane, dichloroethane, ether, diethyl ether, tetrahydrofuran (THF), methyl tert-butyl ether (MTBE), glycol ethers (glyme), dimethoxyethane, bis-2-methoxyethyl ether (diglyme), ester, ethyl acetate, isopropyl acetate, and nitrile (MeCN). When MLP is used as the peptide, the solvent or solvent mixture should be able to provide MLP-acetate or MLP-TFA solubility of ≥30 g/L, ≥50 g/L, or ≥90 g/L based on free MLP.

In some embodiments, the solvent is TFE. The use of TFE as solvent enables conjugation of peptide and chemical agent comprising a reactive moiety at high concentration, ≥50, ≥60, ≥70, ≥80, ≥90, or ≥100 g/L MLP. Further, we have found that MLP and CDM-NAG are stable in TFE. Reacting MLP with CDM-NAG in TFE allows efficient conjugation, with greater that ≥90, ≥95, ≥98, ≥99% primary amine modification, using are little as 1.4-5, 1.4-2.0, 1.4-1.9, 1.4-1.8, 1.4-1.7, 1.4-1.6, or 1.4-1.5 molar equivalents of CDM-NAG.

In some embodiments the solvent is TFE and the peptide is MLP, and the concentration of MLP during the reaction can be 1-100 g/L, 10-100 g/L, 20-100 g/L, 30-100 g/L, 40-100 g/L, 50-100 g/L, or 70-100 g/L. In some embodiments, the concentration of MLP during the reaction can be greater than 100 g/L.

In some embodiments, the solvent is DMSO.

In some embodiments, the CDM-NAG to MLP ratio in the reaction is ≥1.4:1, ≥1.5:1, ≥1.6:1, or ≥1.7:1. In some embodiments, the CDM-NAG to MLP ratio in the reaction is 1.4-5:1, 1.4-2.0:1, 1.4-1.9:1, 1.4-1.8:1, 1.4-1.7:1, 1.4-1.6:1, or 1.4-1.5:1. In some embodiments, the CDM-NAG to MLP ratio in the reaction is 1.5-2:1, 1.5-1.9:1, 1.5-1.8:1, 1.5-1.7:1, or 1.5-1.6:1. In some embodiments, the CDM-NAG to MLP ratio in the reaction is 1.6-2:1, 1.6-1.9:1, 1.6-1.8:1, or 1.6-1.7:1.

The described reaction is optimally run at a reaction temperature of: 10±5° C., 10±4° C. 10±3° C. 10±2° C., or 10±1° C. Any of these temperature ranges provide good reaction rate while keeping side reactions low. We have found that lower temperatures lead to slower reactions. Conversely, we have found that higher temperatures result in increased imide impurity formation.

### Two Step Base Addition

The modified peptide syntheses described herein generally utilize two stages for improved modification (conjugation) efficiency on both N-terminal amines and internal amines (e.g., lysine ε-amines). In some embodiments, the two stages correspond to sequential addition of two different bases. In some embodiments, when a peptide is dissolved in organic solvent as described above, no base addition is required during the first stage. In some embodiments, when other peptides are dissolved in organic solvent as described above, an organic base is added during the first stage. In either case, the first stage is carried out until at least 90%, at least 95%, at least 98%, or at least 99% of N-terminal amines are modified, and a second organic base is added during the second stage.

As used herein, the term “peptide” is meant to include the peptide in any inherently stable form, including in its salt form. For example, in some embodiments, the peptide used in the disclosed methods is a peptide salt, such as a peptide-acetate salt. As used herein, a salt is a chemical compound consisting of an assembly of cations and anions. A peptide salt, for example, is a cationic peptide and associated anion(s). Suitable anions may be selected from, among other things, acetate, conjugate bases of TFA, organic acids, and mineral acids. The methods disclosed herein envision the use of such peptide forms.

In some embodiments, when MLP-acetate (MLP acetate salt) is dissolved in organic solvent as described above, no base addition is required during the first stage. In some embodiments, when MLP-TFA (MLP trifluoroacetate salt) is dissolved in organic solvent as described above, an organic base is added during the first stage. When either MLP-acetate or MLP-TFA is used, a second organic base is added during the second stage.

For the first stage, when necessary, a suitable base is added. Sufficient base is added in amount to control effective pH greater than 5 and less than 9 during the first stage of reaction. The base may be selected from the group comprising: heterocycle, pyridine (Py), pyridine with substitution on the aromatic ring, N-methyl morpholine, N-methyl imidazole, alkylamine, Tris, HEPES, MOPS, triethylamine, and dimethylaminopyridine (DMAP). A suitable amount of the first base is ≥8 equivalents (moles base to moles of peptide). The first stage is optimized to facilitate high N-terminal amine conjugation. The first stage is carried out until at least 90%, at least 95%, at least 98%, or at least 99% of N-terminal amines are modified. In some embodiments, the percent modification of N-terminal amines is determined by reverse phase HPLC.

For the second stage, a second base having a pKa in water >9 is added. In some embodiments, the base is an organic base. In some embodiments, the second base is a trialkylamine such as triethylamine (TEA). A suitable amount of the second base is ≥8 equivalents (moles base to moles of peptide). The second base addition is optimized to facilitate high internal primary amine (lysine ε-amine) conjugation. The second stage is carried out until at least 90% of total primary amines are modified. In some embodiments, the percent modification of total primary amines is determined by TNBS assay.

In some embodiments, we describe herein MLP-NAGₙ synthesis reactions that utilize addition of Py and TEA in two sequential stages. In the first stage, MLP-TFA is dissolved in organic solvent as described above and Py is added. In some embodiments, 8 moles Py is added per to mole MLP in the reaction. Py provides lower basic environment to allow high N-terminal amine conjugation. After conjugation of the N-terminal amine, TEA is added. In some embodiments, 8 moles TEA is added per to mole MLP in the reaction. TEA provides a higher basic environment in which internal primary amine conjugation occurs efficiently. We observed that if only Py was used, internal amine conjugation was low. We further found that if only triethylamine (TEA) was used, N-terminal amine conjugation was lower. Surprisingly, sequential use of both bases significantly improved peptide modification.

In some embodiments, the formed modified peptide is MLP-NAGₙ. In some embodiments, the MLP-NAGₙ conjugate can be a mixture of MLP-NAG₁, MLP-NAG₂, MLP-NAG₃, MLP-NAG₄, and MLP-NAG₅ (i.e. n=1, 2, 3, 4, and 5) with respect to primary amine modification. In some embodiments, the formed MLP-NAGₙ conjugate can be a mixture of MLP-NAG₂, MLP-NAG₃, MLP-NAG₄, and MLP-NAG₅ with respect to primary amine modification. In some embodiments, the formed MLP-NAGₙ conjugate can be a mixture of MLP-NAG₃, MLP-NAG₄, and MLP-NAG₅ with respect to primary amine modification. In some embodiments, the formed MLP-NAGₙ conjugate can be a mixture of MLP-NAG₄, and MLP-NAG₅ with respect to primary amine modification. The described method facilitates at least 90% modification of MLP primary amines.

Using the methods described herein, conjugation of the chemical agent comprising a reactive moiety may further occur at hydroxyl groups or secondary amine groups of the peptide. For example, using the methods described herein, in addition to primary amine modification of a given peptide, the chemical agent comprising a reactive moiety may also be conjugated to amino acid side chains (both natural and unnatural) that comprise hydroxyl groups or secondary amine groups.

In some embodiments, for example, NAG conjugation may further occur at MLP hydroxyl groups or MLP secondary amine groups. Hydroxyl and secondary amine modification can result synthesis of MLP-NAG₆, MLP-NAG₇, MLP-NAG₃, or MLP-NAG₃, or mixtures of these.

Natural and unnatural amino acids may be suitable for inclusion in the peptides that are subject to the methods disclosed herein. Additionally, the amino acids suitable for inclusion in the peptides may be substituted at various positions. The conjugation of a chemical agent comprising a reactive moiety to an amine are well known in the art. Suitable natural and unnatural amino acids that may be modified by a chemical agent comprising a reactive group include, but are not limited to: lysine, arginine, histidine, tryptophan, ornithine, 2,4-diaminobutyric acid, 3-aminoalanine, and the corresponding D-amino acids of same.

### Purification

In some embodiments, the methods described herein comprise precipitation of the modified peptide following the conjugation reaction. In some embodiments, the precipitation may occur by reverse addition into an antisolvent. Suitable antisolvents include, but are not limited to: MeCN, ethyl acetate, acetonitrile, isopropyl acetate, esters, and ethers. In some embodiments, 0.02 vol % TEA (relative to antisolvent) is added to the antisolvent to maintain the solution basic after quench. In some embodiments, carbonate may be added during the precipitation step as a drying agent to further improve stability of the modified peptide. Precipitation of the modified peptide may also allow for the transfer of the conjugate to various manufacturing sites.

In some embodiments, we describe methods of precipitation MLP-NAGₙ following the conjugation reaction. The MLP-NAG conjugate is precipitated by reverse addition into an antisolvent. In some embodiments, the antisolvent can be a solvent in which MLP-NAG solubility is less than 0.5 mg/mL when mixed with trifluoroethanol at a ratio of 9 volumes antisolvent:1 volume TFE containing the MLP-NAG. In some embodiments, 0.02 vol % TEA (relative to antisolvent) is added to the antisolvent to maintain the solution basic after quench, and the MLP-NAG precipitate is then dried under N₂ flow at 0-15° C., 0-10° C., or 0-5° C.

In some embodiments, 3.55 g carbonate (K₂CO₃/NaHCO₃ (3:7 molar ratio)) per gram of MLP is added during the precipitation step act as a drying agent, further improve stability of MLP-NAG and to maintain pH-9.5 of the MLP-NAG upon subsequent reconstitution in water. While not necessary for precipitation, addition of the carbonate improves stability of MLP-NAG during storage and shipment.

In some embodiments, MLP-NAG exhibits greater stability as a precipitate when compared to MLP-NAG in solution. Reverse quench (reaction mixture added to MTBE) and precipitation in MTBE prevents gum solid formation. In addition to aiding in purification and improving stability, precipitation of conjugate solid improves the ability of MLP-NAG to be synthesized at a first manufacturing site and purified and formulated into a final drug product at a second manufacturing site. The dried MLP-NAG precipitate is readily packaged for storage and shipment.

### Tangential Flow Filtration

We describe herein tangential flow filtration (TFF) to exchange the buffer and to remove residual solvent and other small molecules, such as excess chemical agent and hydrolysis products from the modified peptide. Using the methods disclosed herein, the precipitated modified peptide may be dissolved in TFF buffer and dialyzed until desired targeted conductivity, residual solvent level, and chemical agent/peptide ratio are reached. After TFF, the modified peptide solution may be concentrated, dextran added, and vials may be filled and lyophilized.

In some embodiments, the methods disclosed herein provide for TFF to remove the residual solvent, CDM-NAG, and hydrolysis products and other small molecules from the MLP-NAG conjugate. In some embodiments, the precipitated MLP-NAG is dissolved in TFF buffer (Na₂CO₃/NaHCO₃ (3:7 mol), 10-50 mM, pH 9-10) to reach 20-25 g/L MLP concentration. The solution is then dialyzed with TFF buffer until desired targeted conductivity, residual solvent level, and NAG/MLP ratio are reached. After TFF, the MLP-NAG solution is concentrated, dextran is added, the concentration adjusted, vials are filled with a desired amount of MLP-NAG, and solution is optionally lyophilized.

### MLPs

As used herein, a melittin-like peptide comprises a small amphipathic membrane active peptide, comprising about 23 to about 32 amino acids, derived from the naturally occurring bee venom peptide melittin. The naturally occurring melittin contains 26 amino acids and is predominantly hydrophobic on the amino terminal end and predominantly hydrophilic (cationic) on the carboxy terminal end. Melittin-like peptide can be isolated from a biological source or it can be synthetic. A synthetic polymer is formulated or manufactured by a chemical process “by man” and is not created by a naturally occurring biological process. As used herein, melittin-like peptide encompasses the naturally occurring bee venom peptides of the melittin family that can be found in, for example, venom of the species: Apis florea, Apis mellifera, Apis cerana, Apis dorsata, Vespula maculifrons, Vespa magnifica, Vespa velutina, Polistes sp. HQL-2001, and Polistes hebraeus. As used herein, melittin-like peptide also encompasses synthetic peptides having amino acid sequence identical to or similar to naturally occurring melittin peptides. Specifically, melittin-like peptide amino acid sequence encompass those shown in Table 1. In addition to the amino acids substitutions which retain melittin's inherent high membrane activity, 1-8 amino acids can be added to the amino or carboxy terminal ends of the peptide. The list in Table 1 is not meant to be exhaustive, as other conservative amino acid substitutions are readily envisioned. Synthetic melittin-like peptide can contain naturally occurring L form amino acids or the enantiomeric D form amino acids (inverso). The melittin-like peptide amino acid sequence can also be reversed (retro). Retro melittin-like peptide can have L form amino acids or D form amino acids (retroinverso). Melittin-like peptide can have modifying groups, other than masking agents, that enhance tissue targeting or facilitate in vivo circulation attached to either the amino terminal or carboxy terminal ends of the peptide.

In some embodiments, a melittin-like peptide comprises an Apis florea (little or dwarf honey bee) melittin, Apis mellifera (western or European or big honey bee), Apis dorsata (giant honey bee), Apis cerana (oriental honey bee) or derivatives thereof.

In some embodiments, a melittin-like peptide comprises the sequence: Xaa₁-Xaa₂-Xaa₃-Ala-Xaa₅-Leu-Xaa₇-Val-Leu-Xaa₁₀-Xaa₁₁-Xaa₁₂-Leu-Pro-Xaa₁₅-Leu-Xaa₁₇-Xaa₁₈-Trp-Xaa₂₀-Xaa₂₁-Xaa₂₂-Xaa₂₃-Xaa₂₄-Xaa₂₅-Xaa₂₆ wherein:
Xaa₁ is leucine, D-leucine, isoleucine, norleucine, tyrosine, tryptophan, valine, alanine, dimethylglycine, glycine, histidine, phenylalanine, or cysteine,
Xaa₂ is isoleucine, leucine, norleucine, or valine,
Xaa₃ is glycine, leucine, or valine,
Xaa₅ is isoleucine, leucine, norleucine, or valine,
Xaa₇ is lysine, serine, asparagine, alanine, arginine, or histidine,
Xaa₁₀ is alanine, threonine, or leucine,
Xaa₁₁ is threonine or cysteine,
Xaa₁₂ is glycine, leucine, or tryptophan,
Xaa₁₅ is threonine or alanine,
Xaa₁₇ is isoleucine, leucine, norleucine, or valine,
Xaa₁₅ is serine or cysteine,
Xaa₁₀ is isoleucine, leucine, norleucine, or valine,
Xaa₁₁ is lysine or alanine,
Xaa₂₂ is asparagine or arginine,
Xaa₂₃ is lysine or alanine,
Xaa₂₄ is arginine or lysine,
Xaa₂₅ is lysine, alanine, or glutamine,
Xaa₂₆ is optional and if present is glutamine, cysteine, glutamine-NH₂, or cysteine-NH₂; and, and at least two of Xaa₂₁, Xaa₂₃, and Xaa₂₅ are lysine.

In some embodiments, a melittin-like peptide comprises the sequence: Xaa₁-Xaa₂-Xaa₃-Ala-Xaa₅-Leu-Xaa₇-Val-Leu-Xaa₁₀-Xaa₁₁-Xaa₁₂-Leu-Pro-Xaa₁₅-Leu-Xaa₁₇-Ser-Trp-Xaa₂₀-Lys-Xaa₂₂-Lys-Arg-Lys-Xaa₂₆ wherein:
Xaa₁ is leucine, D-leucine, norleucine, or tyrosine,
Xaa₂ is isoleucine, leucine, norleucine, or valine,
Xaa₃ is glycine, leucine, or valine,
Xaa₅ is isoleucine, valine, leucine, or norleucine,
Xaa₇ is lysine, serine, asparagine, alanine, arginine, or histidine,
Xaa₁₀ is alanine, threonine, or leucine,
Xaa₁₁ is threonine, or cysteine,
Xaa₁₂ is glycine, leucine, or tryptophan,
Xaa₁₅ is threonine, or alanine,
Xaa₁₇ is isoleucine, leucine, or norleucine,
Xaa₂₀ is isoleucine, leucine, or norleucine,
Xaa₂₂ is asparagine or arginine, and
Xaa₂₆ is glutamine or cysteine.

In some embodiments, a melittin-like peptide comprises the sequence: Xaa₁-Xaa₂-Gly-Ala-Xaa₅-Leu-Lys-Val-Leu-Ala-Xaa₁₁-Gly-Leu-Pro-Thr-Leu-Xaa₁₇-Ser-Trp-Xaa₂₀-Lys-Xaa₂₂-Lys-Arg-Lys-Xaa₂₆ wherein:
Xaa₁, Xaa₂, Xaa₅, Xaa₁₇ and Xaa₂₀ are independently isoleucine, leucine, or norleucine,
Xaa₁₁ is threonine or cysteine,
Xaa₂₂ is Asparagine or arginine, and
Xaa₂₆ is glutamine or cysteine.

TABLE 1
Melittin-like peptide sequences.

| Seq | Melittin Sequence | Name |
|---|---|---|
| ID | | |
| 1 | GIGAILKVLATGLPTLISWIKNKRKQ | Apis florea |
| 2 | AIGAILKVLATGLPTLISWIKNKRKQ | G1A |
| 3 | CIGAILKVLATGLPTLISWIKNKRKQ | G1C |
| 4 | FIGAILKVLATGLPTLISWIKNKRKQ | G1F |
| 5 | HIGAILKVLATGLPTLISWIKNKRKQ | G1H |
| 6 | IIGAILKVLATGLPTLISWIKNKRKQ | G1I |
| 7 | LIGAILKVLATGLPTLISWIKNKRKQ | G1L |
| 8 | NleIGAILKVLATGLPTLISWIKNKRKQ | G1Nle |
| 9 | VIGAILKVLATGLPTLISWIKNKRKQ | G1V |
| 10 | WIGAILKVLATGLPTLISWIKNKRKQ | G1W |
| 11 | YIGAILKVLATGLPTLISWIKNKRKQ | G1Y |
| 12 | GIGAILKVLACGLPTLISWIKNKRKQ | T11C dMel |
| 13 | GIGAILKVLATLLPTLISWIKNKRKQ | G12L |
| 14 | GIGAILKVLATWLPTLISWIKNKRKQ | G12W |
| 15 | GIGAILKVLATGLPTLISWIKTKRKQ | N22T |
| 16 | YIGAILNVLATGLPTLISWIKNKRKQ | G1Y, K7N |
| 17 | YIGAILAVLATGLPTLISWIKNKRKQ | G1Y, K7A |
| 18 | LIGAILSVLATGLPTLISWIKNKRKQ | G1L, K7S |
| 19 | LIGAILRVLATGLPTLISWIKNKRKQ | G1L, K7R |
| 20 | LIGAILHVLATGLPTLISWIKNKRKQ | G1L, K7H |
| 21 | LIGAILKVLACGLPTLISWIKNKRKQ | G1L, T11C |
| 22 | LIGAILKVLATLLPTLISWIKNKRKQ | G1L, G12L |
| 23 | YIGAILKVLATGLLTLISWIKNKRKQ | G1Y, P14L |
| 24 | LIGAILKVLATGLPCLISWIKNKRKQ | G1L, T15C |
| 25 | LIGAILKVLATGLPTLICWIKNKRKQ | G1L, S18C |
| 26 | YIGAILKVLATGLPTLISAIKNKRKQ | G1Y, W19A |
| 27 | GIGAILKVLACGLPTLISWLKNKRKQ | T11C, I20L |
| 28 | YIGAILKVLATGLPTLISWIANKRKQ | G1Y, K21A |
| 29 | YIGAILKVLATGLPTLISWIKNARKQ | G1Y, K23A |
| 30 | LIGAILKVLATGLPTLISWIKNKAKQ | G1L, R24A |
| 31 | YIGAILKVLATGLPTLISWIKNKRAQ | G1Y, K25A |
| 32 | YIGAILKVLATGLPTLISWIKNKRKC | G1Y, Q26C |
| 33 | LLGAILKVLACGLPTLISWIKNKRKQ | G1L, I2L, T11C |
| 34 | LIGALLKVLACGLPTLISWIKNKRKQ | G1L, I5L, T11C |
| 35 | YIGAILAVLATGLPTLISWIANKRKQ | G1Y, K7A, K21A |
| 36 | YIGAILAVLATGLPTLISWIKNARKQ | G1Y, K7A, K23A |
| 37 | LIGAILKVLACGLPTLLSWIKNKRKQ | G1L, T11C, I17L |
| 38 | LIGAILKVLACGIPTLICWIKNKRKQ | G1L, T11C, S18C |
| 39 | GIGAILKVLACGLPGLIGWIKNKRKQ | T11G, T15G, S18G |
| 40 | GIGAILKVLACGLPALIAWIKNKRKQ | T11A, T15A, S18A |
| 41 | YIGAILAVLATGLPTLISWIANARKQ | G1Y, K7A, K21A, K23A |
| 42 | YIAAILKVLAAALATLISWIKNKRKQ | G1Y, G3A, T11A, G12A, P14A |
| 43 | LLGALLKVLATGLPTLLSWLKNKRKQ | G1L, I2L, I5L, I17L, I20L |
| 44 | LNleGANleLKVLATGLPTLNleSWNleKNKRKQ | G1L, I2Nle, I5Nle, I17Nle, |
| | | I20Nle |
| 45 | LVGAVLKVLATGLPTLVSWVKNKRKQ | G1L, I2V, I5V, I17V, I20V |
| 46 | GLGALLKVLACGLPTLLSWLKNKRKQ | I2L, I5L, T11C, I17L, I20L |
| 47 | GNleGANleLKVLACGLPTLNleSWNleKNKRKQ | I2Nle, I5Nle, T11C, I17Nle, |
| | | I20Nle |
| 48 | CEDDLLLGAILKVLATGLPTLISWIKNKRKQ | CEDDL-Mel G1L, I2L |
| 49 | CLVVLIVVAILKVLATGLPTLISWIKNKRKQ | CLVVL-Mel G1L, I2V, G3V |
| 50 | GIGAVLKVLTTGLPALISWIKRKRQQ | Apis mellifera |
| 51 | CLIGAILKVLATGLPTLISWIKNKRKQ | C-Mel G1L |
| 52 | CNleIGAILKVLATGLPTLISWIKNKRKQ | C-Mel G1Nle |
| 53 | GLIGAILKVLATGLPTLISWIKNKRKQ | G-Mel G1L |
| 54 | LLIGAILKVLATGLPTLISWIKNKRKQ | L-Mel G1L |
| 55 | KLKLIGAILKVLATGLPTLISWIKNKRKQ | KLK-Mel G1L |
| 56 | KLKYIGAILKVLATGLPTLISWIKNKRKQ | KLK-Mel G1Y |
| 57 | CKLKLIGAILKVLATGLPTLISWIKNKRKQ | CKLK-Mel G1L |
| 58 | CKLKNleIGAILKVLATGLPTLISWIKNKRKQ | CKLK-Mel G1Nle |
| 59 | GKLKLIGAILKVLATGLPTLISWIKNKRKQ | GKLK-Mel G1L |
| 60 | CPANLIGAILKVLATGLPTLISWIKNKRKQ | CPAN-dMel G1L |
| 61 | DEPLRAIGAILKVLATGLPTLISWIKNKRKQ | DEPLR-Mel G1A |
| 62 | GIGAILKVLATGLPTLISWIKNKRKQC | Mel-Cys |
| 63 | LIGAILKVLATGLPTLISWIKNKRKQC | G1L Mel-Cys |
| 64 | NleIGAILKVLATGLPTLISWIKNKRKQC | G1Nle Mel-C |
| 65 | LIGAILKVLATGLPTLISWIKNKRKQKLKC | G1L Mel-KLKC |
| 66 | YIGAILKVLATGLPTLISWIKNKRKQPLGIAGQC | G1Y Mel-PLGIAGQC |
| 67 | LIGAILKVLATGLPTLISWIKNKRKQKKKKK | G1L Mel-KKKKK |
| 68 | YIGAILKVLATGLPTLISWIKNKRKQGFKGC | G1Y Mel-GFKGC |
| 69 | CFKLIGAILKVLATGLPTLISWIKNKRKQC | CFK-G1L Mel-C |
| 70 | FGAILKVLATGLPTLISWIKNKRKQ | G1F, I2A |
| 71 | LIGAILKVLATGLPTLISWIKNK | G1L Mel (1-23) |
| 72 | LIGAVLKVLTTGLPALISWIK | G1L, L5V, A10T, T15A Mel (1- |
| | | 23) |
| 73 | LIGAVLKVLTTGLPALISWIKGE | G1L, L5V, A10T, T15A, N22G, |
| | | K23E Mel (1-23) |
| 74 | QKRKNKIWSILTPLGTALVKLIAGIL | G1L retroMel |
| 75 | KLKQKRKNKIWSILTPLGTALVKLIAGIL | G1L retroMel-KLK |
| 76 | GIGAVLKVLTTGLPALISWISRKKRQQ | I5V, A10T, T15A, N22R, R24K, |
| | | K25R Mel-Q |
| 77 | GIGARLKVLTTGLPRISWIKRKRQQ | I5R, A10T, T15R, L16Δ, N22R, |
| | | K25Q |
| 78 | GIGAILKVLSTGLPALISWIKRKRQE | A10S, T15A, N22R, K25Q, |
| | | Q26E |
| 79 | GIGAVLKVLTTGLPALIGWIKRKRQQ | I5V, A10T, T15A, S18G, N22R, |
| | | K25Q |
| 80 | GIGAVLKVLATGLPALISWIKRKRQQ | I5V, T15A, N22R, K25Q |
| 81 | GIGAVLKVLSTGLPALISWIKRKRQQ | I5V, A10S, T15A, N22R, K25Q |
| 82 | GIGAILRVLATGLPTLISWIKNKRKQ | K7R |
| 83 | GIGAILKVLATGLPTLISWIKRKRKQ | N22R |
| 84 | GIGAILKVLATGLPTLISWIKKKKQQ | N22K, R24K, K25Q |
| 85 | GIGAILKVLATGLPTLISWIKNKRKQGSKKKK | Mel-GSKKKK |
| 86 | KKGIGAILKVLATGLPTLISWIKNKRKQ | KK-Mel |
| 87 | GIGAILEVLATGLPTLISWIKNKRKQ | K7E Mel |
| 88 | GIGAVLKVLTTGLPALISWIKRKR | I5V, T15A, N22R, 25-26Δ |
| 89 | GIGAVLKVLTTGLPALISWIKR | I5V, T15A, N22R, 23-26Δ |
| 90 | CIGAVLKVLTTGLPALISWIKRKRQQ | G1C, I5L, T15A, N22R |
| 91 | QQRKRKIWSILAPLGTTLVKLVAGIG | I5V, A10T, T15A, N22R |
| | | retroMel |
| 92 | QQRKRKIWSILAPLGTTLVKLVAGIC | G1C, I5V, A10T, T15A, N22R |
| | | retroMel |
| 93 | QQKKKKIWSILAPLGTTLVKLVAGIC | G1C, I5V, A10T, T15A, N22R, |
| | | R24K retroMel |
| 94 | QKRKNKIWSILTPLGTALVKLIAGIG | Q25K reverse Mel |
| 95 | QQRKRKIWSILAALGTTLVKLVAGIC | G1C, I5V, A10T, P14A, T15A, |
| | | N22R retroMel |

dMel = Melittin-like peptide having D-form amino acids
Nle = norluecine

Unless stated otherwise, weight (mg, g, etc) of MLP or MLP-NAG refers to the weight of the pure MLP component.

The structure of G1L MLP is represented as follows:

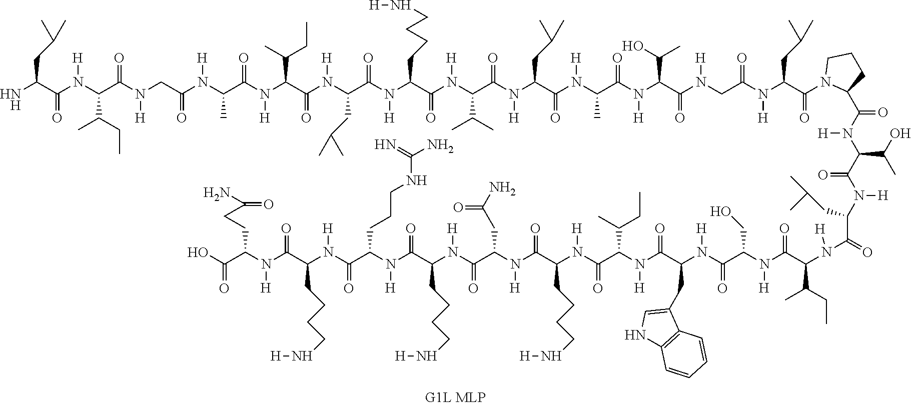

MLP-NAGₙ (also MLP-NAG) is an MLP modified with 1-5 or more CDM-NAG groups. G1L-MLP-NAG is represented as follows:

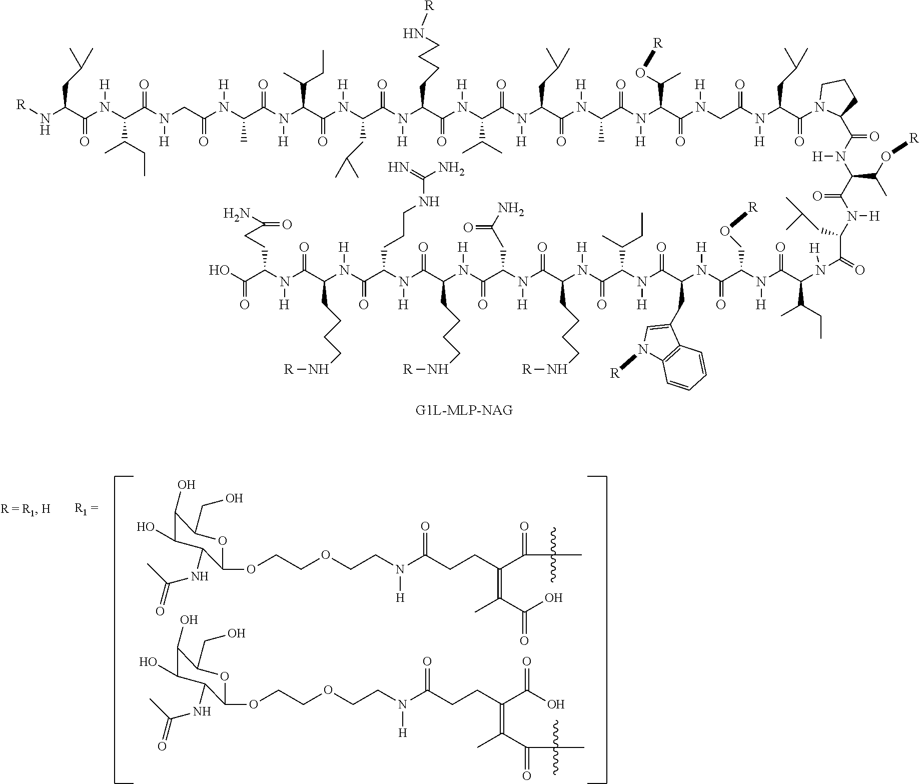

A salt is a chemical compound consisting of an assembly of cations and anions. A peptide salt, for example, is a cationic peptide and associated anion(s). An MLP salt is a cationic MLP and associated anion(s). Suitable anions may be selected from the group comprising: acetate, and the conjugate bases of TFA, organic acids and mineral acids.

### Chemical Agents Comprising a Reactive Moiety or “Modifiers”

The disclosed methods work with various amine-reactive chemical agents capable of modifying the peptide. For example, chemical agents capable of modifying the peptide under the methods disclosed herein include chemical agents that comprise a reactive moiety, in which the reactive moiety is:
(i) an acid (or its activated form, such as an acid chloride, acid anhydride, or ester);
(ii) a ketone or aldehyde;
(iii) an epoxide;
(iv) an alkyl halide;
(v) an activated alcohol; or
(vi) a combination of any of (i) through (v).

The chemical agents comprising a reactive moiety may be hydrophobic or hydrophilic.

In some embodiments, the chemical agent used to modify the peptide comprises an acid anhydride that is a substituted maleic anhydride or a disubstituted maleic anhydride. A substituted maleic anhydride is represented by the structure:

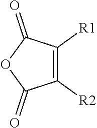

wherein in which R1 comprises a desired group and R2 is a hydrogen (substituted maleic anhydride) or an alkyl group such as a methyl (—CH₃) group, ethyl (—CH₂CH₃) group, or propyl (—CH₂CH₂CH₃) group (disubstituted maleic anhydride).

Suitable disubstituted maleic anhydrides include, but are not limited to:

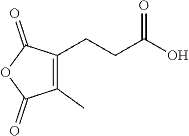

2-propionic-3-methylmaleic anhydride (CDM),

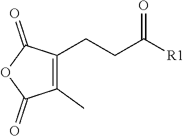

R1 is a desired group
   CDM-R1,

CDM-NAG is a dimethylmaleic anhydride-N-acetylgalactosamine. The structures used in some embodiments of the methods disclosed herein are represented by the following structures:

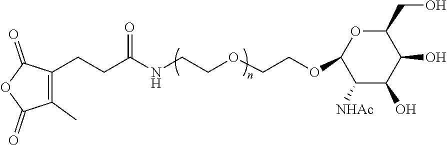

n=1, 2, 3, 4, 5, 6, 7, 8, 9, 10, 11, 12, 13, 14, 15, 16, 17, 18, 19, or 20.

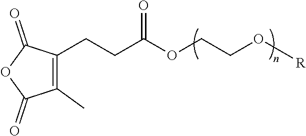

R is a hydrogen, methyl or ethyl, and n is an integer from 2 to 100
   CDM-PEG,

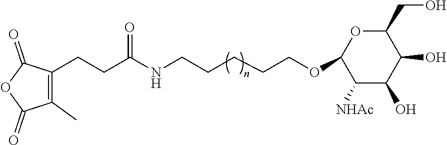

n is a integer from 0 to 6
CDM-NAG (alkyl spacer),

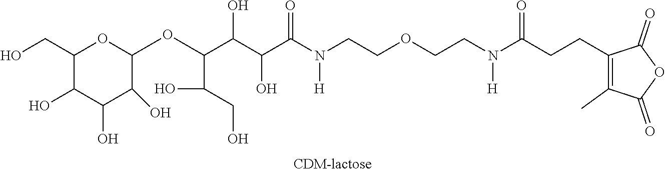 [SMILES: CC1=C(CCC(=O)NCCOCCNC(=O)C(O)C(O)C(OC2OC(CO)C(O)C(O)C2O)C(O)CO)C(=O)OC1=O]

The chemical agent comprising a reactive moiety, which may also be referred to herein as “the modifier,” may also be comprised of one or more desired groups such as targeting group, cell receptor ligand, asialoglycogrotein receptor (ASPGr) ligand, galactose, galactose derivative, N-acetylgalactosamine, carbohydrates, glycans, lactose, saccharides, mannose, mannose derivatives, vitamins, folate, biotin, aptamers, RGD mimics, PEG, steric stabilizers, hapten, label, fluorescent molecule, and the like.

N-acetylgalactosamine is an example of an ASGPr ligand. ASGPr ligands may be selected from the group comprising: lactose, galactose, galactose derivative, N-acetylgalactosamine (GalNAc), galactosamine, N-formylgalactosamine, N-acetyl-galactosamine, N-propionylgalactosamine, N-n-butanoylgalactosamine, and N-iso-butanoyl-galactosamine (Iobst, S. T. and Drickamer, K. J. B. C. 1996, 271, 6686). ASGPr ligands can be monomeric (e.g., having a single galactose derivative) or multimeric (e.g., having multiple galactose derivatives).

In one embodiment, the MLP is reversibly masked by attachment of a chemical agent that comprises an ASGPr ligand, where the chemical agent is conjugated to ≥80% or ≥90% of primary amines on the peptide.

### Aspects of the Disclosed Methods

In some embodiments, we describe a method of synthesis of a peptide comprising the steps, in order:
1) adding the peptide salt in TFE to 2-100, 5-100, 10-100, 20-100, 25-100, 30-100, 40-100, or 50-100 mg/mL peptide and cooling the solution to 5° C., 6° C., 7° C., 8° C., 9° C., 10° C., 11° C., 12° C., 13° C., 14° C., 15° C., 10±5° C., 10±4° C., 10±3° C., 10±2° C., or 10±1° C.;
2) adding a chemical agent comprising a reactive moiety to a concentration of ≥1.0, ≥1.1, ≥1.2, ≥1.3, ≥1.4, ≥1.5, ≥1.6, ≥1.7, ≥1.8, ≥1.9, ≥2, 1.4-5, 1.5-5. 1.6-5, 1.7-5, 1.8-5, 1.9-5, 2-5, 2.5-5, 3-5, 4-5, 1-2, 1.1-2, 1.3-2, 1.4-2, 1.5-2, 1.6-2, 1.7-2, 1.8-2.0, 1.2-1.8, 1.3-1.8, 1.4-1.8, 1.5-1.8, 1.6-1.8, 1.7-1.8, 1.2-1.7, 1.3-1.7, 1.4-1.7, 1.5-1.7, 1.6-1.7, 1.2-1.6, 1.3-1.6, 1.4-1.6, 1.5-1.6, 1.2-1.5, 1.3-1.5, or 1.4-1.5 equivalents (molar ratio of chemical agent to primary amines of the peptide);
3) adding ≥8 equivalents (moles of the first base to moles of the peptide) of a first base and incubating at 5° C., 6° C., 7° C., 8° C., 9° C., 10° C., 11° C., 12° C., 13° C., 14° C., 15° C., 10±5° C., 10±4° C. 10±3° C., 10±2° C., or 10±1° C. until ≥90, ≥91, ≥92, ≥93, ≥94, ≥95, ≥96, ≥97, ≥98, or ≥99% of N-Terminal amines are modified as determined by HPLC; and
4) adding ≥8 equivalents (moles of the second base to moles of the peptide) of a second base and incubating at 5° C., 6° C., 7° C., 8° C., 9° C., 10° C., 11° C., 12° C., 13° C., 14° C., 15° C., 10±5° C., 10±4° C. 10±3° C., 10±2° C., or 10±1° C. until ≥90, ≥91, ≥92, ≥93, ≥94, ≥95, ≥96, ≥97, ≥98, or ≥99% total free amine are modified as determined by HPLC and TNBS assay.

In some embodiments, the peptide is an amphipathic peptide. In some embodiments, the peptide is an MLP. In some embodiments, the chemical agent is CDM-NAG, the first base is Pyridine, and the second base is TEA.

In some embodiments, the method further comprises:
5) adding 1 volume reaction mixture of step 4 to a 9 volumes precooled (0-15° C., 0-10° C., or 0-5° C.), antisolvent such as methyl tertiary butyl ether (MTBE) or acetonitrile (MeCN) (reverse quench). The antisolvent optionally contains TEA (0.02 vol % TEA to MTBE). TEA or other base is added to maintain basic pH during subsequent steps; and
6) adding 3.55 g K₂CO₃/NaHCO₃ (3:7 molar ratio, (optionally both are anhydrous fine powder ≥300 mesh)/g pure peptide, incubating at 0-15° C., 0-10° C., or 0-5° C., filtering to remove solvents, and drying under N₂ at 0-15° C., 0-10° C., or 0-5° C. until dry or nearly dry (10-24 hours).

In some embodiments, the method further comprises:
7) dissolving the precipitated modified peptide in TFF buffer (Na₂CO₃/NaHCO₃ (3:7 mol), 10-50 mM, pH 9-10) at 20-25 g/L peptide, and performing tangential flow filtration.

In some embodiments the method further comprises:
8) concentrating the modified amphipathic peptide solution to about 30 mg/ml, adding dextran to about 10 weight % of the solution weight, filling vials with a desired amount of the modified peptide, and optionally lyophilization of the solution.

In some embodiments, the purified, formulated, modified peptide is stored at 2-8° C. or lower temperature.

In some embodiments we describe a method of synthesis of MLP-NAGₙ comprising the steps, in order:
1) adding MLP salt in TFE to 2-100, 5-100, 10-100, 20-100, 25-100, 30-100, 40-100, or 50-100 mg/mL MLP and cooling the solution to 5° C., 6° C., 7° C., 8° C., 9° C., 10° C., 11° C., 12° C., 13° C., 14° C., 15° C., 10±5° C., 10±4° C., 10±3° C., 10±2° C., or 10±1° C.;
2) adding CDM-NAG to a concentration of ≥1.0, ≥1.1, ≥1.2, ≥1.3, ≥1.4, ≥1.5, ≥1.6, ≥1.7, ≥1.8, ≥1.9, ≥2, 1.4-5, 1.5-5. 1.6-5, 1.7-5, 1.8-5, 1.9-5, 2-5, 2.5-5, 3-5, 4-5, 1-2, 1.1-2, 1.3-2, 1.4-2, 1.5-2, 1.6-2, 1.7-2, 1.8-2.0, 1.2-1.8, 1.3-1.8, 1.4-1.8, 1.5-1.8, 1.6-1.8, 1.7-1.8, 1.2-1.7, 1.3-1.7, 1.4-1.7, 1.5-1.7, 1.6-1.7, 1.2-1.6, 1.3-1.6, 1.4-1.6, 1.5-1.6, 1.2-1.5, 1.3-1.5, or 1.4-1.5 equivalents (molar ratio of CDM-NAG to MLP primary amines).
3) adding ≥8 equivalents (moles Py to moles MLP) Pyridine and incubating at 5° C., 6° C., 7° C., 8° C., 9° C., 10° C., 11° C., 12° C., 13° C., 14° C., 15° C., 10±5° C., 10±4° C. 10±3° C., 10±2° C., or 10±1° C. until ≥90, ≥91, ≥92, ≥93, ≥94, ≥95, ≥96, ≥97, ≥98, or ≥99% of N-terminal amines are modified as determined by HPLC; and
4) adding ≥8 equivalents (moles TEA to moles MLP) TEA and incubating at 5° C., 6° C., 7° C., 8° C., 9° C., 10° C., 11° C., 12° C., 13° C., 14° C., 15° C., 10±5° C., 10±4° C. 10±3° C., 10±2° C., or 10±1° C. until ≥90, ≥91, ≥92, ≥93, ≥94, ≥95, ≥96, ≥97, ≥98, or ≥99% total free amine are modified as determined by HPLC and TNBS assay.

In some embodiments, the method further comprises:
5) adding 1 volume reaction mixture of step 4 to a 9 volumes precooled (0-15° C., 0-10° C., or 0-5° C.), antisolvent such as methyl tertiary butyl ether (MTBE) or acetonitrile (MeCN) (reverse quench). The antisolvent optionally contains TEA (0.02 vol % TEA to MTBE). TEA or other base is added to maintain basic pH during subsequent steps; and
6) adding 3.55 g K₂CO₃/NaHCO₃ (3:7 molar ratio, (optionally both are anhydrous fine powder ≥300 mesh)/g pure MLP, incubating at 0-15° C., 0-10° C., or 0-5° C., filtering to remove solvents, and drying under N₂ at 0-15° C., 0-10° C., or 0-5° C. until dry or nearly dry (10-24 hours).

In some embodiments, the method further comprises:
7) dissolving the precipitated MLP-NAG in TFF buffer (Na₂CO₃/NaHCO₃ (3:7 mol), 10-50 mM, pH 9-10) at 20-25 g/L MLP, and performing tangential flow filtration.

In some embodiments the method further comprises:
8) concentrating the MLP-NAG solution to about 30 mg/ml, adding dextran to about 10 weight % of the solution weight, filling vials with a desired amount of MLP, and optionally lyophilization of the solution.

In some embodiments, the purified, formulated MLP-NAG is stored at 2-8° C. or lower temperature.

The terms synthesis, conjugation, and modification are used interchangeably herein. For purpose of the disclosure herein, the term modifier is used interchangeably with a chemical agent comprising one or more reactive moieties.

The methods described herein can be used to modify a variety of peptides. In some embodiments, the peptides are amphipathic peptides. The methods described herein may also be used to modify biologically-derived molecules or polymers having properties similar to the peptides suitable for use in the disclosed methods.

We have shown that modification of peptide primary amines occurs more readily in an organic solvent in which the peptide, chemical agent comprising a reactive moiety (or modifier), and modified peptide are soluble.

The invention disclosed herein is further illustrated by the following Examples, which should not be construed as limiting.

### EXAMPLES

### Example 1. Synthesis of MLP-NAGₙ

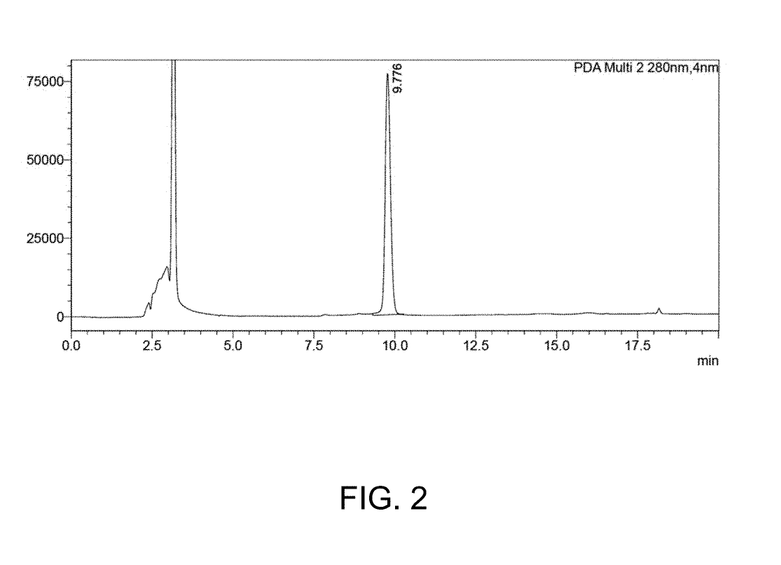

A) First base addition: Peptide MLP (520 mg of MLP-TFA salt, 78 wt % pure MLP to TFA salt) was dissolved in Trifluoroethanol (3.8 mL) and the solution was placed in a 50 mL three neck round-bottom flask, equipped with an overhead stirrer and a thermometer. The solution was cooled to 9-10° C., and CDM-NAG (1.7 eq, 570 mg) was added. Pyridine (40 eq, 455 μL) was then added. The reaction was aged at 10±3° C. until ≥98% N-terminal amine modification was achieved as determine by HPLC, as depicted in FIG. 2:

PDA 280 nm:

| | Ret. Time | |
|---|---|---|
| Peak | (min) | Area % |
| 1 - N-terminal modified MLP | 9.776 | 100 |

B) Second base addition: Triethylamine (TEA) (30 eq, 586 μL) was added and the reaction was aged at 10±3° C. until ≥95% total free amine was modified, as determine by HPLC (3.0% free amine).

### Example 2. Precipitation of MLP-NAG

The reaction mixture from Example 1 was reversely

added to a precooled (0-5° C.), mixed solution of MTBE (38 mL) with Triethylamine (0.75 mL, 0.02 vol % of MTBE). K₂CO₃/NaHCO₃ (3:7 mol, total 1.42 g, anhydrous fine powder ≥300 mesh), was then added and the mixture was aged for 15 min at 0-5° C. The mixture was then filtered and dried under N₂ at 0-5° C. for 30 min to yield a white solid (2.445 g, contained 669 mg MLP-NAG conjugate, 90% yield).

The production of MLP-NAG precipitate using 213 g of MLP free base afforded 1.32 kg of solid after drying. Assay showed MLP was 15.45% of solid.

### Example 3. Analysis of Modification Efficiency

The MLP-NAG precipitate from Example 2 was analyzed for modification efficiency. N-terminal amine modification was 97%, and total free amine modification was 99%. The conjugate solid was stored at −20° C. with desiccant.

| | | |
|---|---|---|
| Free amines by TNBS assay | ≤10% | (0.72-0.81%) |
| Free N-terminal amines | ≤10% | (2.22%) |

MLP-NAGₙ Distribution (Report area %)

| | |
|---|---|
| NAG₆/NAG₅ | 7.077% |
| NAG₅ | 88.623% |
| NAG₄ | 4.300% |

### Example 4. Quantitative Determination of Fraction of Carboxy Dimethylmaleic Anhydride N-Acetylgalactosamine (CDM-NAG) Modification of the N-Terminal Amine of Melittin-Like Peptide (MLP) by Reverse Phase HPLC

a) MLP Reference Standard: 2 mg MLP (from purified powder) was added to 1 ml water to form MLP reference standard. 360 μl of 500 mM MES pH 6.1 solution and 40 μL of 2 mg/mL MLP reference standard were added to an HPLC vial, capped, mixed, and incubated at room temperature for 60 min.

b) MLP-NAG Reference Material Solution: 5 mg MLP-NAG was added to 1 ml water to form MLP-NAG reference material solution. 360 μL of 500 mM MES pH 6.1 solution and 40 μL of MLP-NAG reference material solution were added to an HPLC vial, capped, mixed, and incubated at room temperature for 60 min.

c) MLP-NAG Sample Solution: 5 mg MLP Sample Solution was added to 1 mL water to form MLP-NAG sample solution. 360 μL of 500 mM MES pH 6.1 solution and 40 μL of MLP-NAG sample solution were added to an HPLC vial, capped, mixed, and incubated at room temperature for 60 min

mg/mL MLP or mg/ml MLP-NAG corresponds to the amount of pure MLP peptide. Concentration of MLP or MLP-NAG in a standard or reference was determined by assay of MLP calculated from measurement of a solution's absorbance at 280 nm (MLP assay by UV Absorbance).

d) Blank Solution: 360 μL of 500 mM MES pH 6.1 solution and 40 μL of water were added to an HPLC vial, capped, mixed, and incubated at room temperature for 60 min.

The sample were then analyzed by reverse phase HPLC using the following conditions:

TABLE 2
Chromatographic Parameters: Agilent 1200 HPLC system
with DAD detector; Mobile phase A (0.1% TFA in
water); Mobile phase B (0.1% TFA in ACN).

| Column | Waters XBridge C18, 5 μm, 4.6 mm × | | |
|---|---|---|---|
| | 250 mm, #186003117 | | |
| Run time | 38.0 min | | |
| Detection wavelengths | 280 nm | | |
| Autosampler temperature | 6° C. | | |
| Column temperature | 25° C. | | |
| Flow rate | 1.0 mL/min | | |
| Injection Volume | 20 μL | | |
| Gradient Program | Time (min) | % A | % B |
| | 0.0 | 65 | 35 |
| | 5.0 | 65 | 35 |
| | 25.0 | 0 | 100 |
| | 30.0 | 0 | 100 |
| | 30.5 | 65 | 35 |
| | 38.0 | 65 | 35 |

Retention Time and Relative Retention Time

| | | Relative |
|---|---|---|
| Client | Retention | Retention |
| Compound | Time (min) | Time (RRT) |
| MLP | 14.2 | 0.82 |
| MLP-NAG1 | 17.4 | 1.00 |

For each sample, the peak corresponding to the unconjugated MLP and the peak corresponding to the N-terminus-modified MLP were integrated. The percent area of the N-terminal CDM-NAG modified MLP was determined by dividing the peak area of the N-terminus-modified MLP peak by the sum of the peak area for the N-terminus-modified MLP and unconjugated MLP peak.

### Example 5. Quantitative Determination of Fraction of Free Amines in MLP-NAG by TNBS Assay

0.2 mg/ml, and 0.05 mg/ml stock solutions of purified MLP were made by dissolving in purified water (84.8% MLP by weight). The stock solutions were then diluted into 0.2 M carbonated buffer as indicated in the table for use in generating a standard curve.

| | Volume of MLP | Volume of 0.2M |
|---|---|---|
| MLP Standard | stock (μL) | carbonate buffer (μL) |
| 1 | 0 | 200 |
| 2 | 45 (0.05 mg/mL) | 155 |
| 3 | 90 (0.05 mg/mL) | 110 |
| 4 | 67.5 (0.2 mg/mL) | 132.5 |

A sample solution of 5 mg/ml MLP-NAG in purified water (MLP-NAG solution) was made (i.e. 37 mg/ml MLP-NAG precipitate for MLP-NAG precipitate having 13.5% pure MLP-NAG). 80 μL of MLP-NAG solution was added to 120 μL of 0.2 M carbonate buffer.

mg/mL MLP or mg/ml MLP-NAG corresponds to the amount of pure MLP peptide. Concentration of MLP or MLP-NAG in a standard or reference was determined by assay of MLP calculated from measurement of a solution's absorbance at 280 nm (MLP assay by UV Absorbance).

100 μL of 0.01% 2,4,6-Trinitrobenzenesulfonic acid (TNBS) solution was added to each sample containing 200 μL of standard or sample, mixed, and incubated at room temperature for 30 min. The reactions were quenched by adding 100 μL of 10% SDS solution and then 50 μL of 1N HCl. 200 μL of each reaction solution was transferred to a 96 well plate and absorbance was measured at 335 nm. A standard curve was generated for the MLP standard samples. The concentration of free amines ([MLP]_{observed}) expressed in terms of MLP concentration was calculated using the standard curve.

[MLP]_{observed}=(A335−(Y−intercept))/Slope

An average percentage of free amines was calculated by dividing the observed MLP concentration by the input concentration: % Aminesfree=[MLP]_{observed}÷[MLP]ᵢₙₚᵤₜ×100% where [MLP]ᵢₙₚᵤₜ was the MLP concentration in the MLP-NAG test sample.

### Example 6. MLP Assay by UV Absorbance, Concentration Determination of CDM-NAG-Modified Melittin-Like Peptide (MLP-(CDM-NAG)) by UV Absorbance

In this method, the concentration of samples of the MLP modified by CDM-NAG is determined by UV absorbance at 280 nm. After diluting the samples, the absorbance values are determined and the MLP-(CDM-NAG) concentration is calculated by a formula which includes the dilution factor and the extinction coefficient for MLP-(CDM-NAG). This extinction coefficient is based on the absorbance of the MLP peptide plus five CDMNAG groups bound per peptide molecule, corresponding to a fully modified peptide.

11.76 grams of sodium bicarbonate and 6.36 grams of sodium carbonate were added to a bottle containing 1 l water, stirred until completely dissolved, and filtered through a 0.22 μm PES filter. 4 ml of 0.2 M carbonate buffer was transferred to a 50 ml falcon tube and 36 ml water added. pH was adjusted to pH 9.5±0.2 with 1 M NaOH solution or 1 M HCl solution to form Assay Diluent or no dextran sample blank (20 mM carbonate, pH 9.5). 400 μl of 0.2 M carbonate buffer was transferred to a 15 ml tube and 3.6 ml of water added. 4.0±0.1 mg dextran 1 K was added to form dextran blank. In triplicate, room temperature MLP-(CDM-NAG) solutions were diluted in assay diluent to approximate MLP-(CDM-NAG) concentration of 0.25 mg/ml. (For samples having a nominal MLP-(CDM-NAG) concentration of 5 mg/ml, a dilution factor of 20 was used. For samples having a nominal MLP-(CDM-NAG) concentration of 25 mg/ml, use a dilution factor of 100 was used.) The UV-Vis instrument was set to zero at 280 nm using the appropriate sample blank. The absorbance at 280 nm was then measure for each sample. Samples having absorbance greater than 1.0 were further diluted and measured again. Samples having absorbance less than 0.1 remade at a higher concentration and measured again.
Dilution factor (DF) was calculated as follows:

$$DF = \frac{Weight of MLP \text{-} ( CDM \text{-} NAG ) and assay diluent , g}{Weight of MLP \text{-} ( CDM \text{-} NAG ) , g}$$

MLP-(CDM-NAG) concentration (C) of each sample was determined as follows:

C=[A280*(DF)]/2.16
where, C=concentration in mg/ml, A280=absorbance of sample, and 2.16=the extinction coefficient for MLP-(CDM-NAG).
   Concentration was then averaged for triplicate samples.

### Example 7. Quantitative Determination of Fraction of Carboxy Dimethylmaleic Anhydride N-Acetylgalactosamine (CDM-NAG) Modification of the N-Terminal Amine of Melittin-Like Peptide (MLP) by Reverse Phase HPLC

a) MLP Reference Standard: 2 mg MLP (from purified powder) was added to 1 ml water to form MLP reference standard. 360 μl of 500 mM MES pH 6.1 solution and 40 μL of 2 mg/mL MLP reference standard were added to an HPLC vial, capped, mixed, and incubated at room temperature for 60 min.

b) MLP-NAG Reference Material Solution: 5 mg MLP-NAG was added to 1 ml water to form MLP-NAG reference material solution. 360 μL of 500 mM MES pH 6.1 solution and 40 μL of MLP-NAG reference material solution were added to an HPLC vial, capped, mixed, and incubated at room temperature for 60 min.

c) MLP-NAG Sample Solution: 5 mg MLP Sample Solution was added to 1 mL water to form MLP-NAG sample solution. 360 μL of 500 mM MES pH 6.1 solution and 40 μL of MLP-NAG sample solution were added to an HPLC vial, capped, mixed, and incubated at room temperature for 60 min

For each of the above, mg/mL MLP corresponds to the amount of pure MLP peptide. Percent MLP or MLP-NAG in a standard or reference was determined by assay of MLP calculated from measurement of a solution's absorbance at 28 nm (AM-C14110779-A-10: MLP assay by UV Absorbance).

d) Blank Solution: 360 μL of 500 mM MES pH 6.1 solution and 40 μL of water were added to an HPLC vial, capped, mixed, and incubated at room temperature for 60 min.

The sample were then analyzed by reverse phase HPLC using the following conditions:

TABLE 3
Chromatographic Parameters: Agilent 1200 HPLC system
with DAD detector; Mobile phase A (0.1% TFA in
water); Mobile phase B (0.1% TFA in ACN).

| Column | Waters XBridge C18, 5 μm, 4.6 mm × | | |
|---|---|---|---|
| | 250 mm, #186003117 | | |
| Run time | 38.0 min | | |
| Detection wavelengths | 280 nm | | |
| Autosampler temperature | 6° C. | | |
| Column temperature | 25° C. | | |
| Flow rate | 1.0 mL/min | | |
| Injection Volume | 20 μL | | |
| Gradient Program | Time (min) | % A | % B |
| | 0.0 | 65 | 35 |
| | 5.0 | 65 | 35 |
| | 25.0 | 0 | 100 |
| | 30.0 | 0 | 100 |
| | 30.5 | 65 | 35 |
| | 38.0 | 65 | 35 |

Retention Time and Relative Retention Time

| | | Relative |
|---|---|---|
| Client | Retention | Retention |
| Compound | Time (min) | Time (RRT) |
| MLP | 14.2 | 0.82 |
| MLP-NAG1 | 17.4 | 1.00 |

For each sample, the peak corresponding to the unconjugated MLP and the peak corresponding to the N-terminus-modified MLP were integrated. The percent area of the N-terminal CDM-NAG modified MLP was determined by dividing the peak area of the N-terminus-modified MLP peak by the sum of the peak area for the N-terminus-modified MLP and unconjugated MLP peak.

### Example 8. Identification and Determination of Relative Abundance of Various Species of MLP-NAGₙ

Standard MLP-NAGₙ solution (STD): 6 mg/mL MLP-NAGₙ standard in water.

Sensitivity solution: 0.006 mg/mL MLP-NAGₙ standard in water.

Sample solution: 6 mg/mL (MLP equivalent) MLP-NAGₙ sample in water.

TABLE 4
Chromatographic Parameters: Agilent G1969A TOP-MS with
DAD detector; Mobile phase A and Diluent: (0.1%
NH₃•H₂0 in water); Mobile phase B: Acetonitrile

| Column | Waters Xbridge Cl8, 3.0 mm × 100 mm × | | |
|---|---|---|---|
| | 3.5 μm | | |
| Run time | 30 min | | |
| Detection wavelengths | 280 nm | | |
| Autosampler temperature | 6° C. | | |
| Column temperature | 30° C. | | |
| Flow rate | 1.0 mL/min | | |
| Injection Volume | 10 μL | | |
| Gradient Program | Time (min) | % A | % B |
| | 0.0 | 90 | 10 |
| | 1.5 | 80 | 20 |
| | 16.5 | 35 | 65 |
| | 19.5 | 5 | 95 |
| | 22 | 5 | 95 |
| | 22.1 | 90 | 10 |
| | 30.0 | 90 | 10 |
| Diluent | 0.1% NH₃•H₂0 in purified water (v/v) | | |
| Needle wash | purified water | | |
| | Ion Source | | |
| Mass Spec parameters | Dual ESI | Ion Polarity | Negative |
| Gas Temperature | 300° C. | drying gas | 5 L/min |
| VCap | 3500 V | fragmentor | 50 V |

A multi-charged mass spectrum (MS) of MLP-NAG was obtained. Deconvolution and derivation of the MS Abundance Table was performed using Agilent MassHunter Workstation Acquisition Software (TOF/Q-TOF B.02.00 of equivalent). MS peaks representing the isotope peaks of MLP-NAG and their relative abundance from the MS Abundance Table were selected. Average Molecular Weight (MW) was calculated as follows:

Average MW=Sum(MASSi×Abundance % i)

where
MASSi=Monoisotopic mass
Abundance %=Abundance of each monoisotopic mass divided by sum of all monoisotopic mass abundances

Identification of various species of MLP-NAGₙ by their masses:

| MLP-NAGₙ | Expected Average MW |
|---|---|
| n = 3 | 4298.05 |
| n = 4 | 4772.76 |
| n = 5 | 5247.85 |
| n = 6 | 5722.05 |
| n = 7 | 6196.25 |
| n = 8 | 6671.61 |

The relative abundance of various species of MLP-NAGₙ by the area percent of each peak was calculated. Relative percent free amine by the area percent (in the extract ion chromatograms) of various species of MLP-NAGₙ was calculated as follows:

$$Free amine \% = \frac{{NAG}_{4} Area \%}{5} + \frac{{NAG}_{3} Area \% \times 2}{5} + \frac{{NAG}_{2} Area \% \times 3}{5} + \frac{{NAG}_{1} Area \% \times 4}{5}$$

MLP-NAGₙ distribution by area percent (in the extract ion chromatograms) of various species was calculated as follows:

Area %=A_{C}/A_{T}100%

where:
A_{C} represented the peak area of each species in sample solution
A_{T} represented the total peak area from sample chromatograms

### Example 9. Larger Scale Synthesis of MLP-NAG

Pure MLP (1.11 kg, 1.0 eq) in 1.45 kg of MLP-TFA salt (76.3 wt %) was dissolved in TFE (14.3 kg). The solution was cooled to 10° C., CDM-NAG (1.63 kg, 8.5 eq) was added, then pyridine (1.21 kg, 40 eq), and the mixture was aged at 10±3° C. The reaction was monitored by HPLC to ensure ≤2.0% N-terminal amine remained unmodified before proceeding to the next step. The In Process Control (IPC) analysis showed 1.4% N-terminal free amine left. Triethylamine (TEA, 1.17 kg, 30 eq) was then added to the reaction and aged at 10±3° C. The reaction was monitored by HPLC to ensure ≤5.0% total free primary amines remained unmodified before proceeding to the next step. The IPC analysis showed 3.3% free amine left. The reaction was finally quenched by reverse addition of reaction solution into a mixture of t-butyl methyl ether (MTBE, 80 kg), TEA (1.55 kg), K₂CO₃ (1.65 kg) and NaHCO₃ (2.34 kg). The solid was filtered, washed with MTBE, and dried under nitrogen to yield MLP-NAG Precipitate 6.66 kg.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of modifying amines of a peptide under non-aqueous conditions with a chemical agent that comprises a reactive moiety, the method comprising:
(a) dissolving the peptide and the chemical agent in an organic solvent;
(b) adding a first base; and
(c) incubating under non-aqueous conditions until at least 90% of the N-terminal amines of the peptide are modified.

2. The method of claim 1, wherein the method further includes:
(d) adding a second base after 90% of the N-terminal amines have been modified under non-aqueous conditions.

3. The method of claim 2, wherein part (d) of the method further comprises incubating until at least 90% of the total primary amines of the peptide are modified with the chemical agent under non-aqueous conditions.

4. The method of claim 3, wherein the peptide is an amphipathic peptide.

5. The method of claim 3, wherein the organic solvent is selected from the group consisting of: trifluoroethanol (TFE), hexafluoro-iso-propanol (HFIP), dimethyl sulfoxide (DMSO), mixtures of TFE, HFIP, and DMSO, mixtures of TFE, HFIP, or DMSO with one or more miscible solvents.

6. The method of claim 3, wherein step (a) further comprises a miscible solvent selected from the group consisting of: alcohol, methanol (MeOH), ethanol, (EtOH), n-propanol, n-butanol, ethylene glycol, dimethylformamide (DMF), dimethylacetamide (DMAc), N-methyl-2-pyrrolidone (NMP), halogenated solvent, chloroform, dichloromethane, dichloroethane, ether, diethyl ether, tetrahydrofuran (THF), methyl tert-butyl ether (MTBE), glycol ethers (glyme), dimethoxyethane, bis-2-methoxyethyl ether (diglyme), ester, ethyl acetate, isopropyl acetate, and nitrile (MeCN).

7. The method of claim 3, wherein the organic solvent is TFE.

8. The method of claim 3, wherein the first base is added and the first base is heterocycle, pyridine (Py), pyridine with substitution on the aromatic ring, N-methyl morpholine, N-methyl imidazole, alkylamine, Tris, HEPES, MOPS, triethylamine, or dimethylaminopyridine (DMAP).

9. The method of claim 8, wherein the first base is pyridine.

10. The method of claim 3, wherein the second base is an organic base.

11. The method of claim 10, wherein the second base is a trialkylamine.

12. The method of claim 11, wherein the trialkylamine is triethylamine.

13. The method of claim 3, wherein the peptide is MLP.

14. The method of claim 13, wherein the MLP is selected from any of SEQ ID NOs: 1-95.

15. The method of claim 3, wherein the reactive moiety of the chemical agent comprises (i) an acid (or its activated form, such as an acid chloride, acid anhydride, or ester); (ii) a ketone or aldehyde; (iii) an epoxide; (iv) an alkyl halide; (v) an activated alcohol; or (vi) a combination of any of (i) through (v).

16. The method of claim 15, wherein the chemical agent comprises a substituted maleic anhydride.

17. The method of claim 15, wherein the chemical agent comprises an asialoglycoprotein receptor ligand.

18. The method of claim 15, wherein the chemical agent comprises the structure represented by: 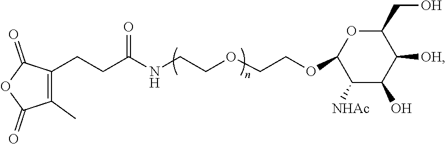
wherein n is an integer from 1-20.

19. The method of claim 15, wherein the chemical agent comprises a polyethylene glycol.

20. The method of claim 19, wherein the chemical agent comprises the structure represented by: 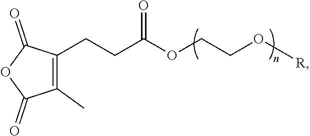
wherein n is an integer from 2 to 100 and R is hydrogen, methyl, or ethyl.

21. The method of claim 3, wherein the ratio of chemical agent to peptide is ≥1.4:1; ≥1.5:1; ≥1.6:1; ≥1.7:1; 1.4-5:1; 1.4-2.0:1; 1.4-1.9:1; 1.4-1.8:1; 1.4-1.7:1; 1.4-1.6:1; 1.4-1.5:1; 1.5-2:1; 1.5-1.9:1; 1.5-1.8:1; 1.5-1.7:1; 1.5-1.6:1; 1.6-2:1; 1.6-1.9:1; 1.6-1.8:1; or 1.6-1.7:1.

22. The method of claim 3, wherein the peptide is single stranded and between about 5 and about 40 amino acids, or the peptide is a dimer or is double stranded and is between about 5 and about 80 amino acids.
